SEQUENCE LISTING (7 sequences)

SEQ ID NO: 1 - DNA, 72 bp, Artificial Sequence
  Description of Artificial Sequence Synthetic oligonucleotide
ggcaggaagacaaacacgatgggggggtatgatttgatgtggttgttgcatgatcgtggtctgtggtgctgt

SEQ ID NO: 2 - DNA, 25 bp, Artificial Sequence
  Description of Artificial Sequence Synthetic oligonucleotide
agaaaaaaaaacgcaaaaaaaaaaa

SEQ ID NO: 3 - DNA, 72 bp, Artificial Sequence; identical to SEQ ID NO: 1
  Description of Artificial Sequence Synthetic oligonucleotide

SEQ ID NO: 4 - DNA, 25 bp, Artificial Sequence
  Description of Artificial Sequence Synthetic
agaaaaaaaaacgcaaaaaaaaaaa

SEQ ID NO: 5 - DNA, 21 bp, Artificial Sequence
  Description of Artificial Sequence Synthetic probe
tgcggcatgaaccggaggccc

SEQ ID NO: 6 - DNA, 21 bp, Artificial Sequence
  Description of Artificial Sequence Synthetic oligonucleotide
taatgcatgtcacaggcggga

SEQ ID NO: 7 - DNA, 20 bp, Artificial Sequence
  Description of Artificial Sequence Synthetic oligonucleotide
gggcctccggttcatgccgc

(19) United States
(12) Patent Application Publication
(10) Pub. No.: US 2013/0281315 A1
(43) Pub. Date: Oct. 24, 2013

(54) ENHANCING SURFACE PLASMON RESONANCE IMAGING SIGNAL

(71) Applicants: MARINELLA G. SANDROS, GREENSBORO, NC (US); VINCENT C. HENRICH, GREENSBORO, NC (US); STEPHEN A. VANCE, SOPHIA, NC (US)

(72) Inventors: MARINELLA G. SANDROS, GREENSBORO, NC (US); VINCENT C. HENRICH, GREENSBORO, NC (US); STEPHEN A. VANCE, SOPHIA, NC (US)

(73) Assignee: THE UNIVERSITY OF NORTH CAROLINA AT GREENSBORO, GREENSBORO, NC (US)

(21) Appl. No.: 13/868,148

(22) Filed: Apr. 23, 2013

Related U.S. Application Data
(60) Provisional application No. 61/637,324, filed on Apr. 24, 2012.

(51) Int. Cl. C12Q 1/68; G01N 33/566

(52) U.S. Cl. CPC C12Q 1/682; G01N 33/566
USPC 506/9; 530/391.3

(57) ABSTRACT

Described is a biointerface using near-infrared quantum dots for surface plasmon resonance imaging biosensors.

### CROSS-REFERENCE TO RELATED APPLICATIONS

This application claims benefit of priority to U.S. Provisional Patent Application No. 61/637,324 to Sandros et al., entitled “ENHANCING SURFACE PLASMON RESONANCE IMAGING SIGNAL,” filed Apr. 24, 2012 which is incorporated herein by reference in its entirety.

### REFERENCE TO SEQUENCE LISTING

The instant application contains a Sequence Listing which has been submitted in ASCII format via EFS-Web and is hereby incorporated by reference in its entirety. Said ASCII copy, created on Apr. 17, 2013, is named 44483.01.0002_SL.txt and is 2,358 bytes in size.

### BACKGROUND

1. Field of the Invention

The present invention relates to surface plasmon resonance (SPR) techniques.

2. Related Art

One limitation of previous SPR resonance techniques is that the techniques have a detection limit in the low nanomolar range.

### SUMMARY

According to a first broad aspect, the present invention provides a method comprising the following steps: (a) detecting binding of a streptavidin-coated quantum dot to an array of surface biomolecules to thereby produce detected binding results, and (b) displaying the detected binding results to a user and/or saving the detected binding results to a storage medium, wherein each surface biomolecule comprises an ssDNA hybrid that is bound to a gold substrate and a biotin-tagged ssDNA complementary sequence hybridized to a thiol-modified ssDNA probe sequence, wherein the streptavidin-coated quantum dot binds to the biotin-tagged ssDNA complementary sequence, and wherein step (a) comprises using surface plasmon resonance imaging on the gold substrate to detect the binding of the streptavidin-coated quantum dot to the array of surface biomolecules.

According to a second broad aspect, the present invention provides a method comprising the following steps: (a) detecting binding of a plurality of ssDNA-protein-quantum dot complexes to an array of thiol-modified ssDNA probe sequences to thereby produce detected binding results, and (b) displaying the detected binding results to a user and/or saving the detected binding results to a storage medium, wherein the thiol-modified ssDNA probe sequences are bound to a gold substrate, wherein each ssDNA-protein-quantum dot complex comprises: a streptavidin-coated quantum dot, one or more biotin-tagged ssDNA complementary sequences bound to respective streptavidin-coated quantum dots of the one or more streptavidin-coated quantum dots, wherein each of the ssDNA-protein-quantum dot complexes binds to a respective thiol-modified ssDNA probe sequence of the array of thiol-modified ssDNA probe sequences, and wherein step (a) comprises using surface plasmon resonance imaging on the gold substrate to detect the binding of the plurality of ssDNA-protein-quantum dot complexes to the array of thiol-modified ssDNA probe sequences.

According to a third broad aspect, the present invention provides a composition comprising: a capture antibody complex, an antigen bound to the capture antibody complex, and a biotinylated-detection antibody complex bound to the antigen.

According to a fourth broad aspect, the present invention provides a method comprising the following steps: (a) detecting binding of a plurality of biomarkers to an array of surface biomolecules to thereby produce detected binding results, and (b) displaying the detected binding results to a user and/or saving the detected binding results to a storage medium, wherein each surface biomolecule comprises a calixcrown ProLinker B bound to a gold substrate, wherein each biomarker comprises: a capture antibody complex, and an antigen bound to the capture antibody complex, wherein a biotinylated-detection antibody complex is bound to the antigen, wherein a streptavidin-coated quantum dot is bound to the biotinylated-detection antibody complex, and wherein step (a) comprises using surface plasmon resonance imaging on the gold substrate to detect the binding of the plurality of biomarkers to the array of surface biomolecules.

According to a fifth broad aspect, the present invention provides a method comprising the following steps: (a) detecting binding of a plurality of biomarkers to an array of surface biomolecules to thereby produce detected binding results, and (b) displaying the detected binding results to a user and/or saving the detected binding results to a storage medium, wherein each surface biomolecule comprises a thiolated polyethylene glycol bound to a gold substrate, wherein each biomarker comprises: a capture antibody complex, and an antigen bound to the capture antibody complex, wherein a biotinylated-detection antibody complex is bound to the antigen, wherein a streptavidin-coated quantum dot is bound to the biotinylated-detection antibody complex, and wherein step (a) comprises using surface plasmon resonance imaging on the gold substrate to detect the binding of the plurality of biomarkers to the array of surface biomolecules.

According to a sixth broad aspect, the present invention provides a method comprising the following steps: (a) detecting binding of an avidin-coated quantum dot to an array of surface biomolecules to thereby produce detected binding results, and (b) displaying the detected binding results to a user and/or saving the detected binding results to a storage medium, wherein each surface biomolecule comprises an ssDNA hybrid that is bound to a gold substrate and a biotin-tagged ssDNA complementary sequence hybridized to a thiol-modified ssDNA probe sequence, wherein the avidin-coated quantum dot binds to the biotin-tagged ssDNA complementary sequence, and wherein step (a) comprises using surface plasmon resonance imaging on the gold substrate to detect the binding of the avidin-coated quantum dot to the array of surface biomolecules.

According to a seventh broad aspect, the present invention provides a method comprising the following steps: (a) detecting binding of a plurality of ssDNA-protein-quantum dot complexes to an array of thiol-modified ssDNA probe sequences to thereby produce detected binding results, and (b) displaying the detected binding results to a user and/or saving the detected binding results to a storage medium, wherein the thiol-modified ssDNA probe sequences are bound to a gold substrate, wherein each ssDNA-protein-quantum dot complex comprises: an avidin-coated quantum dot, one or more biotin-tagged ssDNA complementary sequences bound to respective avidin-coated quantum dots of the one or more avidin-coated quantum dots, wherein each of the ssDNA-protein-quantum dot complexes binds to a respective thiol-modified ssDNA probe sequence of the array of thiol-modified ssDNA probe sequences, and wherein step (a) comprises using surface plasmon resonance imaging on the gold substrate to detect the binding of the plurality of ssDNA-protein-quantum dot complexes to the array of thiol-modified ssDNA probe sequences.

According to an eighth broad aspect, the present invention provides a method comprising the following steps: (a) detecting binding of a plurality of biomarkers to an array of surface biomolecules to thereby produce detected binding results, and (b) displaying the detected binding results to a user and/or saving the detected binding results to a storage medium, wherein each surface biomolecule comprises a calixcrown ProLinker B bound to a gold substrate, wherein each biomarker comprises: a capture antibody complex, and an antigen bound to the capture antibody complex, wherein a biotinylated-detection antibody complex is bound to the antigen, wherein an avidin-coated quantum dot is bound to the biotinylated-detection antibody complex, and wherein step (a) comprises using surface plasmon resonance imaging on the gold substrate to detect the binding of the plurality of biomarkers to the array of surface biomolecules.

According to a ninth broad aspect, the present invention provides a method comprising the following steps: (a) detecting binding of a plurality of biomarkers to an array of surface biomolecules to thereby produce detected binding results, and (b) displaying the detected binding results to a user and/or saving the detected binding results to a storage medium, wherein each surface biomolecule comprises a thiolated polyethylene glycol bound to a gold substrate, wherein each biomarker comprises: a capture antibody complex, and an antigen bound to the capture antibody complex, wherein a biotinylated-detection antibody complex is bound to the antigen, wherein an avidin-coated quantum dot is bound to the biotinylated-detection antibody complex, and wherein step (a) comprises using surface plasmon resonance imaging on the gold substrate to detect the binding of the plurality of biomarkers to the array of surface biomolecules.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration of an SPRi measurement system according to one embodiment of the present invention.

FIG. 2 is a plot representation of the SPRi kinetic signal after the addition of MUC-1 peptide to MUC-1 aptamer (target) and bare gold (non-target, control).

FIG. 3 shows in schematic form a comparison of: (1) the separation distance between a gold surface and streptavidin QDs bound to single-stranded DNA, (2) the separation distance between a gold surface and streptavidin QDs bound to PSA-ACT sandwich complexes bound to calixcrown ProLinker B, and (3) the separation distance between a gold surface and aptamer-coated nanoparticles bound to biomarkers that are bound to aptamers.

FIG. 4 is a schematic representation of a method of detecting DNA hybridization according to one embodiment of the present invention.

FIG. 5 is a schematic representation of a method of detecting DNA hybridization according to one embodiment of the present invention.

FIG. 6 is a plot representation of the surface plasmon resonance imaging (SPRi) kinetic signal after the addition of sandwich quantum dots (SA-QDs) to 50-biotin-tagged double-stranded DNA (dsDNA) target (solid) and nontargeted dsDNA (dashed), and the corresponding difference curve (dotted).

FIG. 7 is a plot representation of the SPRi kinetic signal after the addition of ssDNA-QD complex to thiol-modified ssDNA target sequence (solid) and nontargeted sequence (dashed), and the corresponding difference curve (dotted).

FIG. 8 is a plot representation of the SPRi kinetic signal after the addition of bare NIR QDs to 50-biotin-tagged dsDNA target (black, solid) nontargeted dsDNA (dashed), and the corresponding difference curve (dotted).

FIG. 9 is a plot representation comparison of the SPRi kinetic signal after the addition of 525, 705, 780, and 800 nm SA-QDs to 50-biotin-tagged double-stranded DNA (dsDNA) target.

FIG. 10 shows SPRi kinetic curves for the detection of DNA hybridization using a sandwich assay for various ssDNA target concentrations.

FIG. 11 shows concentration gradient curves for the detection of DNA hybridization using a sandwich assay for various ssDNA target concentrations.

FIG. 12 is a three-dimensional top view fluorescence image of an SPRi biochip after introduction of sandwich quantum dots (SA-QDs).

FIG. 13 is a three-dimensional side view fluorescence image of the SPRi biochip of FIG. 12.

FIG. 14 is a schematic representation of gold chip functionalization with calixcrown ProLinker B followed by capture antibody PSA-ACT complex, PSA-ACT antigen, biotinylated detection antibody PSA-ACT complex, and streptavidin-coated QDs.

FIG. 15 is plot of SPRi kinetic curves for various concentrations of PSA-ACT antigen.

FIG. 16 is a plot of concentration gradient curves for various concentrations of PSA-ACT antigen of FIG. 15.

FIG. 17 is a schematic representation of gold chip functionalization with PEG-COOH and PEG-OH followed by the addition of capture antibody PSA-ACT complex, PSA-ACT antigen, biotinylated detection antibody PSA-ACT complex, and streptavidin-coated QDs.

FIG. 18 is a plot of SPRi kinetic curves for detection of PSA-ACT in spiked serum.

FIG. 19 are difference images corresponding to the curves of FIG. 18 showing time-lapsed binding kinetics for initial buffer injection, PSA-ACT complex in spiked serum injection, buffer wash, dAb-biotin injection, SA-QD injection, and final buffer wash on three spots of anti-PSA (left) and anti-IgG (right).

FIG. 20 is a plot of SPRi kinetic curves for detection of C-reactive protein (CRP) in in 10 mM Tris buffer containing 50 mM NaCl and 2 mM CaCl₂.

FIG. 21 is a plot of SPRi kinetic curves for detection of CRP in spiked serum.

FIG. 22 shows difference images corresponding to the curves of FIG. 21 showing time-lapsed binding kinetics before injection of CRP and after injection of Apt-UCNs.

### DETAILED DESCRIPTION OF THE PREFERRED EMBODIMENTS

### Definitions

Where the definition of a term departs from the commonly used meaning of the term, applicant intends to utilize the definitions provided below, unless specifically indicated.

For purposes of the present invention, it should be noted that the singular forms, “a,” “an” and “the,” include reference to the plural unless the context as herein presented clearly indicates otherwise.

For purposes of the present invention, a value or property is “based” on or “derived” from a particular value, property, the satisfaction of a condition or other factor if that value is derived by performing a mathematical calculation or logical decision using that value, property, condition or other factor.

For purposes of the present invention, the term “aptamer” refers to a single-stranded oligonucleic acid molecule or peptide molecule that binds to a particular target molecule.

For purposes of the present invention, the term “array” refers to a one-dimensional or two-dimensional set of surface biomolecules. An array may be any shape.

For purposes of the present invention, the term “biomarker” refers to any organic or inorganic molecule that indicates the physiological, behavioral, cellular, chemical, cognitive or biological status of the organism. Diagnostic use of a biomarker may result from its presence or its absence, alone or in combination with other molecules or conditions for a given embodiment.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “biotinylated biomarker” refers to a biomarker that is biotinylated. An example of a biotinylated biomarker is a biomarker having bound thereto a biotinylated-detection antibody complex.

For purposes of the present invention, the term “biosensor” refers to a device for detection of an analyte that includes a biological component (includes tissue, microorganism, cell receptors, enzymes, antibodies, nucleic acid, molecules extracted from biological sources and elements that can be created by biological engineering) and a physicochemical detector component.

For purposes of the present invention, the term “camera” refers to any type of camera or other device that senses light intensity. Examples of a camera include a digital camera, a scanner, a charged-coupled device (CCD), a complementary metal oxide semiconductor (CMOS) sensor, a photomultiplier tube, an analog camera such as film camera, etc. A camera may include additional lenses and filters.

For purposes of the present invention, the term “capture antibody complex” and the term refers to a complex for capturing or detecting an antibody. An example of a capture antibody complex is the capturing or detecting antibody to human Prostate Specific Antigen/Alpha-1-Antichymotrypsin complex (PSA/ACT complex).

For purposes of the present invention, the term “detected binding results” refer to the results and/or data obtained and/or produced based on the detection of a detected species binding to something else, such as a substrate, an array of surface biomolecules, an array of thiol-modified ssDNA probe sequences, etc. Examples of detected species include: a biomolecule, a biomolecule coated on a quantum dot, such as a streptavidin-coated quantum dot, an avidin-coated quantum dot, etc., quantum dot complexes, such as ssDNA-protein-quantum dot complexes, biomarkers, etc.

For purposes of the present invention, the term “nanoparticles” refers to any material with a size range between 2 to 100 nm. Upconverting nanoparticles are an example of the type of nanoparticles that may be used in various embodiments of the present invention.

For purposes of the present invention, the term “near-infrared quantum dot (NIR QD) refers to a quantum dot that emits near-infrared light.

For purposes of the present invention, the term “near real-time” refers to results obtained within 5 minutes of sample injection into the detecting instrument. In one embodiment, the present invention allows for near real-time measurements of biomolecules binding to surface biomolecules on a substrate.

For purposes of the present invention, the term “quantum dot” refers to a special class of semiconducting material composed of periodic group elements II-VI, III-V, IV-VI with a size range between 2 to 10 nm.

For purposes of the present invention, the term “small molecule” refers to a molecule that has a molecular weight of 200 Da or less.

For purposes of the present invention, the term “storage” and the term “storage medium” refer to any form of storage that may be used to store bits of information. Examples of storage include both volatile and non-volatile memories such as MRRAM, ERAM, flash memory, floppy disks, Zip™ disks, CD-ROM, CD-R, CD-RW, DVD, DVD-R, hard disks, optical disks, etc. A storage medium may be a piece of paper, plastic, etc. with printing on it.

For purposes of the present invention, the term “surface biomolecule” refers to any biomolecule bound to a surface of a substrate. The binding of a target biomolecule to a surface biomolecule may allow for the SPR measurements on the substrate for the binding of the target biomolecule to the surface biomolecule. Aptamers are an example of the type of surface biomolecules that may be used in various embodiments of the present invention.

For purposes of the present invention, the term “upconverting nanoparticles” refers to nanoparticles that convert low-energy radiation to higher-energy emissions.

For purposes of the present invention, the term “thiol-modified” refers an aptamer that has a thiol incorporated into the aptamer. The thiol may be incorporated in the aptamer during the synthesis of the thiol-modified aptamer.

For purposes of the present invention, the term “visual display device” or “visual display apparatus” includes any type of visual display device or apparatus such as a CRT monitor, LCD screen, LEDs, a projected display, a printer for printing out an image such as a picture and/or text, etc. A visual display device may be used to display results of the methods of the present invention to a user. A visual display device may be a part of another device such as a computer monitor, television, projector, cell phone, smartphone, laptop computer, tablet computer, handheld music and/or video player, personal data assistant (PDA), handheld game player, head-mounted display, a heads-up display (HUD), a global positioning system (GPS) receiver, automotive navigation system, dashboard, watch, microwave oven, electronic organ, automated teller machine (ATM), etc.

### Description

In one embodiment, the present invention employs aptamer-functionalized nanoparticles to enhance the sensitivity of SPRi for the ultrasensitive detection of TBI protein biomarkers. SPR measurements on a planar gold film are now well established as the leading label-free alternative to fluorescence-based methods for the investigation of biomolecular interactions. One limitation of SPR is the detection limit in the low nanomolar range. Efforts to improve sensitivity through the use of spherical gold nanoparticles conjugated to a secondary biomolecular probe as part of a sandwich assay have been reported by several groups. For example, spherical DNA-functionalized gold nanoparticles have been used to evoke a 100-1000 increase in sensitivity resulting in detection limits in the 1-10 pM range. In one embodiment, the present invention provides an ultrasensitive SPRi detection method using streptavidin-coated near-infrared quantum dots (NIR QDs) for the direct detection of DNA and proteins. In one embodiment of the present invention it is possible to directly measure single-stranded DNA oligonucleotides within minutes at femtomolar concentrations using the NIR QDs in conjunction with SPRi. This provides a 25-fold enhancement compared to conventional SPRi measurements of the same surface assay format in the absence of NIR QDs.

It is hypothesized this signal enhancement by the quantum dots is caused by the QDs' mass-loading effect and spontaneous emission coupling with propagating surface plasmons. Owing to the success with NIR QDs in enhancing SPRi signal detection, it may be possible to use upconverting nanoparticles (UCNs) to increase the surface plasmon coupling between the gold surface and the nanoparticles. Near infrared (NIR)-to-visible up-conversion nanomaterials can convert NIR light to visible light upon NIR light radiation. This phenomenon has been observed in the transition metal, lanthanide, and actinide ions, though the highest efficiencies are found in lanthanide-doped materials. To date, the highest upconversion efficiencies observed have been in hexagonal phase NaYF₄ bulk materials doped with the Er³⁺/Yb³⁺ or Tm³⁺/Yb³⁺ ion couples, synthesized via solid-state methods. Upconversion materials show superior photostability, as the lanthanide ions responsible for their fluorescence emission are doped within the nanocrystal core such that they are well shielded from the surroundings. The ability to suspend UCNs as clear colloidal solutions has opened the door for their use in several new technologies, including their use as labels in biological assays and imaging. In some embodiments, the present invention provides methods with 1 centimolar detection sensitivity for detecting biomolecules in solution. In some embodiments, the present invention provides methods with attomolar detection sensitivity for detecting biomolecules in solution.

In one embodiment of the present invention, a surface plasmon resonance imaging chip biointerface uses near infrared (NIR) quantum dots (QDs) for the enhancement of surface plasmon resonance imaging (SPRi) signals in order to extend their application for medical diagnostics. The measured SPRi detection signal following the QD binding to the surface was amplified 25-fold for 1 nM concentration of single-stranded DNA (ssDNA) and 50-fold for 1 μg/mL concentration of prostate-specific antigen (PSA), a cancer biomarker, thus substantiating their wide potential to study interactions of a diverse set of small biomolecules. This significant enhancement is attributed to the QDs' mass-loading effect and spontaneous emission coupling with propagating surface plasmons and, which allow the SPRi limit of detection to be reduced to 100 fM and 100 pg/mL for ssDNA and PSA, respectively. Furthermore, SPRi may be easily integrated with fluorescent imaging for advanced correlative surface-interaction analysis.

Quantum dots (QDs) have appealing crucial roles in quantum information and nanophotonics,¹⁻⁷ and communications between surface plasmons (SPs) and QDs represent a subject of great interest. Recently, Wei et al. observed that the interaction between QDs and propagating SPs in silver nanowires were bidirectional.⁸ Propagating SPs were able to excite excitons which resulted in QD emission and, by direct excitation of QDs, the exciton decay was converted to propagating SPs. This bidirectional relationship between QDs and SPs has not yet been explored extensively with surface-sensitive systems such as SPR1.

SPRi has emerged as a versatile proteomic and genomic tool for in situ real-time biosensing.⁹⁻¹⁴ The SPRi detection method takes place through a thin metal layer (gold or silver, 50 nm) that coats the surface of a prism where it couples the incident light to the surface plasmons by evanescent waves.¹⁵ As opposed to classical scanning angle or wavelength SPR systems, SPRi technology provides a CCD camera which detects variation in reflected light (differential image) and a sensogram that measures changes in reflectivity of p-polarized light at a fixed angle. The combination of the two components allows for visualization of the entire biochip surface in real time and the means to monitor multiple interactions continuously and simultaneously.

Current studies focus mainly on detecting interactions between large biomolecules because small molecules generate a small angular shift and only a small fractional reflectivity change at the sensing surface. For medical diagnostic applications, biological samples often contain attomolar or femtomolar concentrations of biomolecules (i.e., DNA) which is well below the nanomolar detection limit of SPRi. Increasing the sensitivity and improving the SPRi bio-interface in order to generate bigger signals has therefore been an active area of research. One approach involves the addition of higher molecular weight molecules that selectively bind to the receptor/analyte adduct. Mass enhancement by a factor of 4 was first demonstrated for the DNA hybridization reaction through the addition of streptavidin and biotinylated DNA.¹⁶ Hu et al. combined mass and enzyme-based enhancement for the detection of Vascular Endotheliol Growth Factor (VEGF) reaching a limit of detection (LOD) of 1 pM under physiological conditions.¹⁷

The integration of nanoscale materials with SPRi technology that leads to new methods with unique capabilities has also been explored. For instance, gold nanoparticles bound to DNA have been extensively used for SPRi signal amplification. In concert with the immobilized peptide nucleic acid sequences, gold nanoparticle labels enabled the detection of point mutations with a low 1 fM LOD using microfluidics.¹⁸ Likewise, Fang et al. used polythiamine modified-gold nanoparticles that bind to a polyadenine tail to enhance the detection of microRNAs (LOD 10 fM).¹⁹ Most of these approaches were, however, application-specific and typically required an enzymatic reaction in addition to gold nanoparticle tags and long incubation periods (4 to 24 hrs.) to reach fM detection limits. Conversely, an SPRi enhancement has also been reported for functionalized silica-coated gold nanorods to amine-modified single-stranded DNA.²° While this approach can be used for a variety of biomarkers, the SPRi enhancement generated a 28% change in reflectivity using DNA concentrations in the lower mM range. Integrated surface corrugations such as gold gratings or nanopillars provided electromagnetic enhancements, however, the detection sensitivity was limited to 100 pM.²¹⁻²² In one embodiment the present invention extends the application of SPRi sensors for medical diagnostics by taking advantage of the NIR QDs' intrinsic physical and optical properties.

Only a few studies have explored QDs with SPR biosensing. In one particular report, DNA was functionalized to red-emitting QDs and introduced to the complementary strand on the surface of the chip, yielding a 20-nM LOD with a modest change in percent reflectivity (%ΔR˜0.3-0.4).²³ Similarly, with pre-functionalized QDs to the sensor chip to monitor the binding of proteins, LOD remained in the nM range.²⁴ Recently, Golub et al. engineered a sophisticated SPR-based aptasensor by functionalizing gold nanoparticles or CdS quantum dots with a subunit of the anticocaine aptamer.²⁵ Even though their approach demonstrated great versatility by detecting cocaine using multiple systems such as electrochemical, photoelectrochemical and SPR techniques, their LOD was down to only 10⁻⁶ M.

In one embodiment, the present invention provides an ultrasensitive SPRi biosensor surface for the detection of DNA and proteins by developing a surface-specific coating in combination with NIR QDs' mass loading effect and coupling to propagating surface plasmons. The SPRi signal is largely amplified, providing femtomolar (fM) LOD after small molecules binding within a short period of reaction time (<10 min) Furthermore, the exploitation of NIR QDs gives rise to the possibility of combining and translating the event of biomolecules binding onto the chemically modified gold surface with 3D fluorescent imaging.

In one embodiment of the present invention, a QD₈₀₀-amplified SPRi method may be used to lower the limits of detection of ssDNA from 10⁻⁹ M to 10⁻¹⁵ M and PSA-ACT breast cancer biomarkers from 10⁻⁶ g/mL to 10⁻¹⁰ g/mL. These dramatic enhancements in SPRi signals are believed to be due, in part, to the resonant coupling that occurs between the QDs' spontaneous emission with the propagating SPs and also partly due to the mass-loading effect of SA-QDs.

In one embodiment of the present invention, the QD signal amplification may be used to study interactions of a wider selection of small biomolecules which are currently unattainable with the SPRi instrument. The ability to detect genomic DNA with very low detection limits in a very short period of reaction time should accelerate its application in the fields of genetic testing, as well as bacterial and viral recognition. In addition, this methodology facilitates the integration of fluorescence imaging without the need of any additional surface labeling, thus providing the means for advanced correlative surface-interaction analysis.

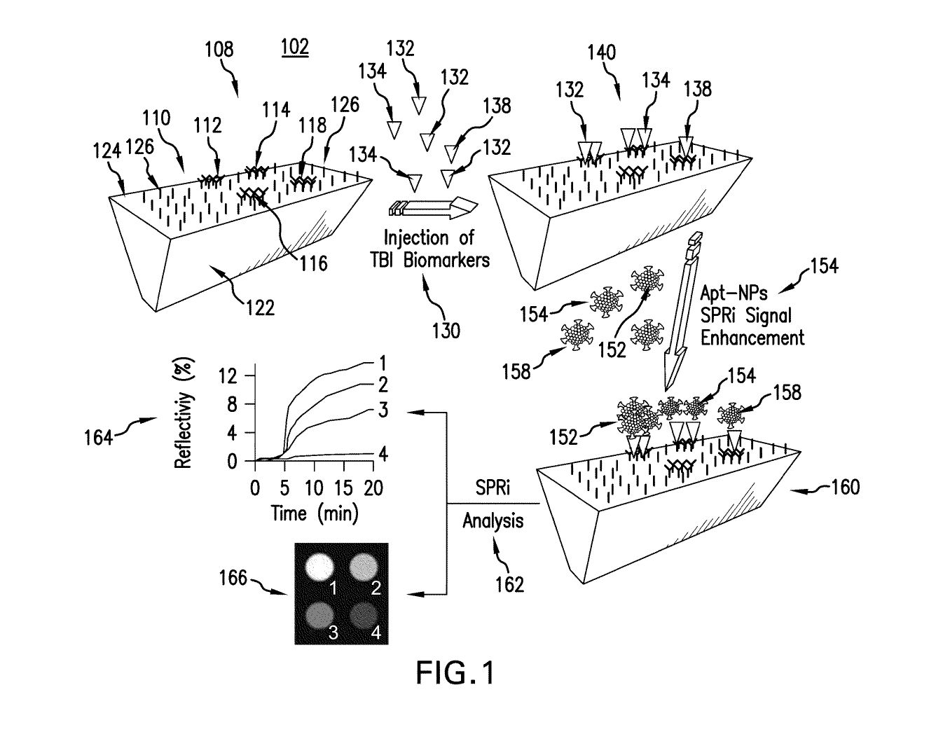

FIG. 1 is a schematic illustration of an SPRi measurement system 102 according to one embodiment of the present invention. At stage 108, SPRi measurement system 102 provides an SPRi gold-coated prism 110, functionalized with thiolated aptamers 112, 114, 116 and 118 that bind specifically to S100B, GFAP and UCH-L1 and NSE, respectively. SPRi gold-coated prism 110 includes a glass prism 122, a gold coating 124 on glass prism 122 and Denhardt's thiolated aptamers 112, 114, 116 and 118 bind to gold surface 126 Followed by blocking the surface with 1×Denhardt's solution and polyethylene glycol methyl ether thiol 126 applied on top of gold coating 124. At step 130 of SPRi measurement system 102, TBI biomarkers s100b 132, GFAP 134 and NSE 138 are injected into a flow cell (not shown) containing SPRi gold-coated prism 122 resulting in TBI biomarkers S100B 132, GFAP 134 and NSE 136 binding to aptamers 112, 114 and 118, respectively, of SPRi gold-coated prism 110 at stage 140. At step 150, aptamer-coated nanoparticles (Apt-NPs) 152, 154 and 158 are injected into a flow cell (not shown) containing SPRi gold-coated prism 122, resulting in Apt-NPs 152, 154 and 158 binding to TBI biomarkers s100b 132, GFAP 134 and NSE 138, respectively at stage 160. At step 162, SPRi measurements are collected in the form of a sensogram 164 and a difference image 166 generated from a CCD camera (not shown in FIG. 1). s100b (S100 calcium binding protein B) is a protein of the S-100 protein family. GFAP (glial fibrillary acidic protein) is a protein that in humans is encoded by the GFAP gene. UCH-L1 (ubiquitin carboxy-terminal hydrolase L1) is a deubiqutinating enzyme. NSE (neuron specific enolase) is an enzyme in humans that is encoded by the ENO2 gene.

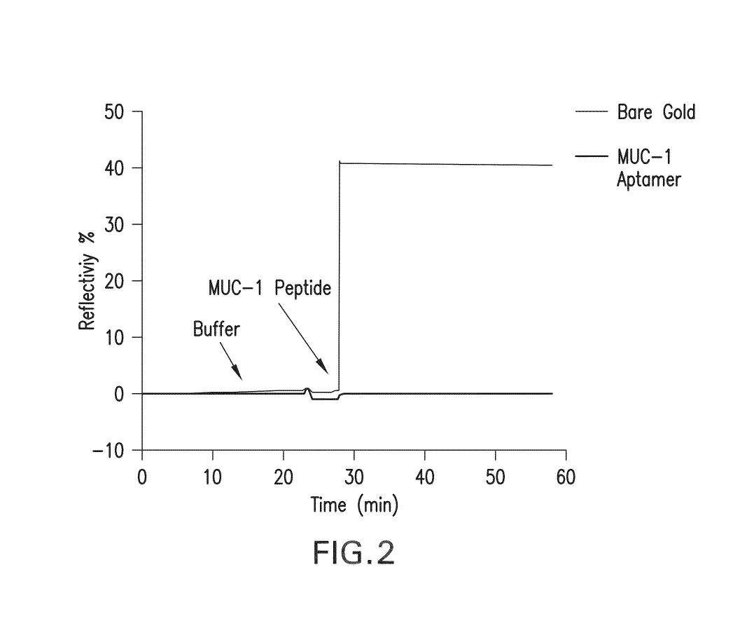

FIG. 2 is a plot representation of the SPRi kinetic signal after the addition of MUC-1 peptide to MUC-1 aptamer (target) and bare gold (non-target, control).

Many of the above-described techniques of the present invention are described in L. Malic, M. G. Sandros, M. Tabrizian, “Designed biointerface using near-Infrared quantum dots for ultrasensitive surface plasmon resonance imaging biosensors,” Anal. Chem., 83, 5222-29 (2011), the entire contents and disclosure of which are incorporated herein by reference.

Although the use of streptavidin-coated near-infrared quantum dots is described in various embodiments of the present invention described above and in the embodiments of the present invention described below in the examples, avidin-coated near-infrared quantum dots may also be used in place of streptavidin-coated near-infrared quantum dots in embodiments of the present invention.

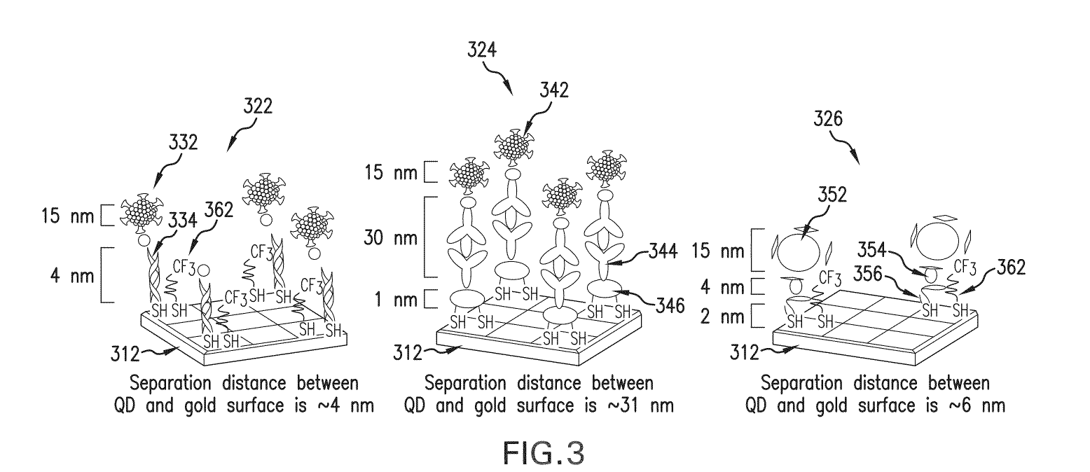

FIG. 3 shows a comparison of the separation distance between a gold surface 312 for three detection system, i.e., detection systems 322, 324 and 326. In detection system 322, streptavidin QDs 332 is bound to single-stranded DNA 334 with a separation distance of 4 nm. In detection system 324, streptavidin QDs 342 bound to PSA-ACT sandwich complexes 344 bound to calixcrown ProLinker B 346 with a separation distance of 31 nm. In detection system 326, aptamer-coated nanoparticles 352 are bound to biomarkers 354 that are bound to aptamers 356 with a separation distance of 6 nm. A larger enhancement is observed in detection system 322 as opposed to detection system 324. This is also related to the fact that the separation distance between QDs and gold surface in detection system 324 is much larger than in detection system 322. In detection system 326, there is a small separation distance between gold and nanoparticles and therefore, in some embodiments of the present invention, may provide a better system for detecting PSA-ACT antigen the detection system 324. In detection system 322, HDFT 362 is bound to gold surface 312 for the purpose of minimizing non-specific binding. In detection system 326, HDFT 362 is bound to gold surface 312 for the purpose of minimizing non-specific binding. In each of detection systems 322, 324 and 326 the streptavidin QDs have diameters of about 15 to 20 nm.

### EXAMPLES

### Materials and Methods

Chemicals and reagents used in the examples described below include: Streptavidin (BioChemika), BSA (Albumin from bovine serum), Human Serum from human male AB plasma, Immunoglobulin G from human serum (BioChemika), HDFT (Heptadecafluoro-1-decanethiol, Fluka), NHS(N-Hydroxysuccinimide, Fluka), ETH (Ethanolamine hydrochloride), potassium phosphate dibasic solution, 1M, pH 8.9 (1M K2HPO4), Tris-EDTA buffer solution, pH 8.0 (TE buffer), phosphate buffer saline (PBS, pH 7.4), sodium chloride (NaCl), sodium hydroxide (NaOH), Dimethyl sulfoxide and ethanol were all purchased from Sigma-Aldrich (St. Louis, Mo., USA). HBS buffer (0.01 M HEPES, pH 7.4, 0.15 M NaCl, 3 mM EDTA, 0.005% Surfactant P20) was purchased from GE Healthcare. all oligonucleotide (ODN) sequences²⁶ were obtained from Integrated DNA Technologies (Coralville, Iowa, USA). PEG-OH (Hydroxy-terminated hexa(ethylene glycol) undecane Thiol (OH)), PEG-COOH (Carboxyl-terminated hexa(ethylene glycol) undecane Thiol (COOH)) and PEG-Biotin (Biotinylated tri(ethylene glycol) hexadecane Thiol (Biotin)) were purchased from Nanoscience Instruments, USA. Calixcrown (ProLinker B) was purchased from Proteogen, Korea. Human Prostate-Specific Antigen, PSA/ACT COMPLEX Monoclonal Antibodies (capture antibody cAb A45520259P and detection antibody dAb A45530259P) and antigen (1-029-ACT, 90 kDa, PSA 28 KDa and ACT 60-65 KDa) were purchased from Biospacific, USA. EZ-link Sulfo-NHC-LC-LC Biotin and Zeba Spin columns (Pierce) were purchased from Fisher, Canada. Streptavidin-coated quantum dots (15 to 20 nm in size), Qdot 800 STVD, Qdot 525 STVD and Qdot 705 STVD were purchased from Invitrogen, USA, and APC-eFluor 780 conjugated streptavidin QDs were purchased from Ebiosciences, USA.

### Surface Functionalization

Substrates (gold-coated prism, GenOptics, France) were cleaned in piranha and surface functionalization was performed according to previously reported procedures for DNA probe²¹ and capture antibody immobilization (Calixcrown, PEG).²⁷⁻²⁸ Briefly, DNA immobilization was performed using 1 μM thiol-modified 20-mer oligonucleotide probe sequence comprising a dT spacer (5′-/HS-C6/-dT-GCG GCA TGA ACC GGA GGC CC-3′) (SEQ ID NO: 5) in 1 M KH₂PO₄ for 180 min. Following the immobilization, substrates were treated with 20 mM HDTF for 10 minutes to render the probes highly accessible to the target while preventing unspecific target-binding to the gold surface as a control, a thiol-modified random 20-mer oligonucleotide sequence (5′-/HSC6/-dT-AAT GCA TGT CAC AGG CGG GA-3′) (SEQ ID NO: 6) was immobilized in the same manner on the SPR substrate.

The capture antibody immobilization was performed using two different thiol-based surface chemistries, Calixcrown and mixed monothiols, in order to attach functional groups for antibody immobilization. The Calixcrown surface chemistry relies on surface functionalization with ProLinker B Calixcrown derivative that contains thiol moiety that binds to the gold surface. The chip was prepared by soaking the surface of the gold-coated prism in 3 mM ProLinker Bin CHCl₃ solution for 1 hour and rinsing sequentially with CHCl₃, acetone, ethanol, and deionized water. The surface was dried under a stream of nitrogen and exposed to a solution of 100 μg/mL cAb in PBS for 3 hours. Finally, the surface was blocked with BSA (10 mg/ml, in PBS) for 1 hour.

Mixed monothiol surface chemistry is based on gold-surface functionalization with thiolated PEG-OH and PEG-COOH mixture. The surface was immersed overnight in 1 mM mixed monothiol solution in ethanol (PEG-COOK and PEG-OH at a ratio of 4:1). The surface was then rinsed in ethanol, dried under a stream of nitrogen and followed by a 30-minute activation of amine groups with NHS/EDC (50 mM/200 mM). Finally, cAb was immobilized for 1 hour at a concentration of 100 μg/mL in PBS by spotting three 2 μl, droplets on the surface, followed by a deactivation of amine group by 1 M ethanolamine for 10 min.

### Antibody Biotinylation

The experimental steps for biotinylating detection antibodies (dAb), purifying the biotinylated dAb, and measuring the level of biotin incorporation were carried out using the EZ-link sulfo-NHS-LC-Biotinylation Kit according to the manufacturer's protocol.

### SPR Measurements

SPRi detection of biomolecular-binding interactions was performed using the SPRi Lab+ apparatus equipped with an 800 nm LED source, CCD camera and a flow cell (GenOptics, France), placed in Memmert Peltier-cooled incubator (Rose Scientific, Canada) for temperature control (for detailed system specifications, see L. Malic, B. Cui, M. Tabrizian, T. Veres, “Nanoimprinted plastic substrates for enhanced surface plasmon resonance imaging detection,” Opt. Express, 17, 20386-92 (2009)). The entire biochip surface was imaged during the angular scan, while for each experiment three ˜500 μm diameter spots were selected for the monitoring of the binding interactions with both the probe and the control. For each spot, the reflected intensity was displayed as a function of angle in the plasmon curve diagram. The slope of the plasmon curves was automatically computed to facilitate the selection of the working angle for kinetic analysis, which corresponded to the point of the plasmon curve at which the slope was maximized. In kinetic analysis, the reflected intensity (% R) was traced as a function of time showing the kinetics of the binding events that take place at the surface of the chip. The reported curves were averaged over three spots and each experiment has been repeated at least three times. Unless otherwise noted, the plots of the reflectivity curves represent the difference curves taken as the difference between the target and control curves. The control curve plots are also included for the lowest concentration of the target in order to facilitate the computation of the detection limit. The SPRi difference images captured by the CCD camera were recorded to obtain real-time information about the reactions occurring at the surface of the chip. The binding event was seen as an increase in the reflected intensity, characterized by a bright spot, which is easily distinguishable from the dark background. For each experiment, a peristaltic pump with injection loop was connected to the flow cell and used to control sample flow at a rate of 50 μl/min.

DNA hybridization experiments were carried out using a biotin-modified 20-mer complementary oligonucleotide target sequence (3′-CGC CGT ACT TGG CCT CCG GG-5′-biotin) (SEQ ID NO: 7) in 1 M NaCl in TE buffer (hybridization buffer). Hybridization kinetic curves were obtained at room temperature for all elements of the array simultaneously. A baseline signal was obtained first for the hybridization buffer, followed by the hybridization signal for which targets were injected sequentially into the flow cell of the SPRi, allowing the target to bind to the immobilized probe for 10 minutes. Following the hybridization signal, streptavidin-conjugated QDs at a concentration of 5 nM in hybridization buffer solution were injected for 10 minutes allowing them to bind to the biotinylated target. Finally, the substrate was washed with buffer solution and the difference in the reflected intensity (% ΔR) was computed by taking the difference between the initial and final buffer signals for both, the target (% ΔR_{target}) and the QDs (% ΔR_{QDs}). Successive hybridizations were followed by surface regeneration using 50 mM NaOH.

Sandwich immunoassay was performed using PSA-ACT complex antigen and biotinylated dAb in HBS buffer. A baseline signal was obtained first for the HBS buffer, followed by the antibody-antigen binding signal for which proteins were injected sequentially into the flow cell of the SPRi, allowing them to bind to the immobilized cAb for 10 minutes. Following the protein binding, biotinylated dAb was injected into the flow cell at a concentration of 20 μg/mL in HBS buffer for 10 minutes. Subsequently, streptavidin-conjugated QDs, 5 nM in concentration in HBS buffer solution were injected for 10 minutes allowing them to bind to the biotinylated dAb. Finally, the substrate was washed with buffer solution and the difference in the reflected intensity (% ΔR) was computed by taking the difference between the initial and final buffer signals, similarly to that performed for the DNA hybridization.

The signal enhancement (SE) is computed by taking the ratio between the SPRi signal of the streptavidin-coated QDs and that of the biotinylated target (SE=% ΔR_{QDs}/% ΔR_{target}). The reported limit of detection (LOD) represents the minimum detectable target concentration for which the SPR signal (% ΔR) is at least three times higher than that of the control.

### Example 1

To investigate the level of the SPRi signal amplification provided by the NIR QDs, both a sandwich and direct detection assay were performed to uncover which method is more sensitive.

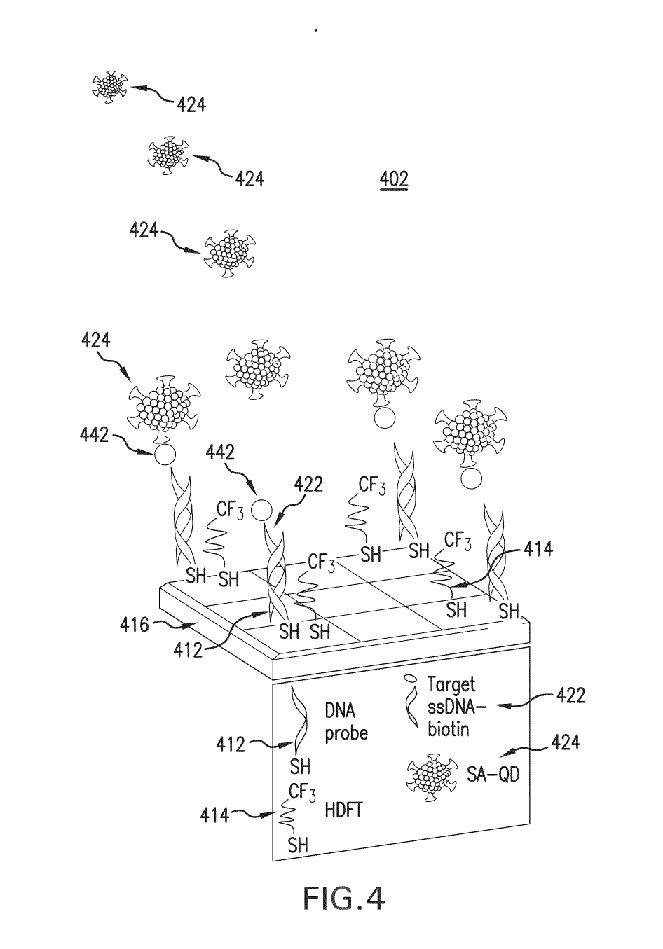

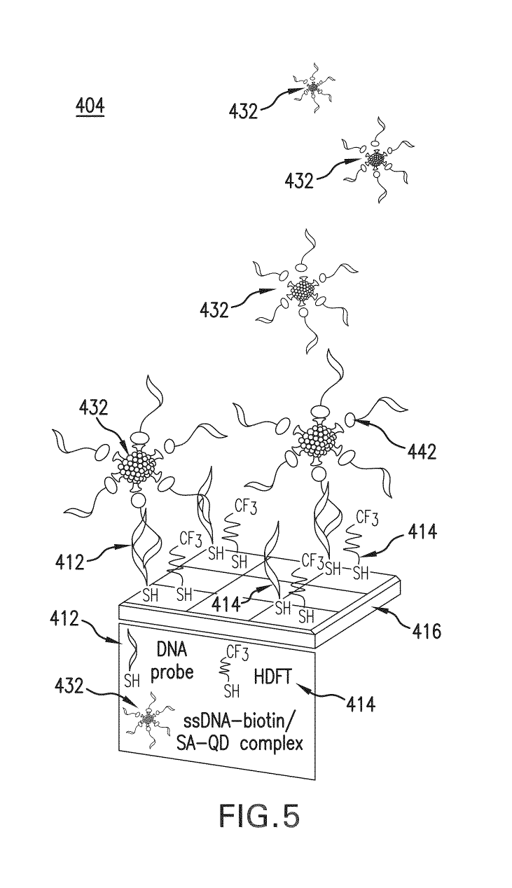

FIGS. 4 and 5 show schematic representations of two systems, 402 and 404, respectively, of the present invention implemented for the detection of DNA hybridization. In both systems 402 and 404, a initially thiol-modified ssDNA probe sequence 412 and HDFT 414 are functionalized onto a gold surface 416. FIG. 4 shows system 402 that involves (1) adding 50-biotin-tagged ssDNA complementary sequences 422 to gold surface 416 and then (2) adding SA-QDs 424 to 50-biotin-tagged ssDNA complementary sequences 422.

FIG. 5 shows system 404 that involves the direct addition of ssDNA-QD complex 432 a 50-biotin-tagged ssDNA complementary sequence 422 attached to SA-QDs 424, i.e., each 50-biotin-tagged ssDNA complementary sequence 422 includes biotin 442. In FIG. 5, ssDNA-QD complex 432 is tagged multiple times with biotin 442.

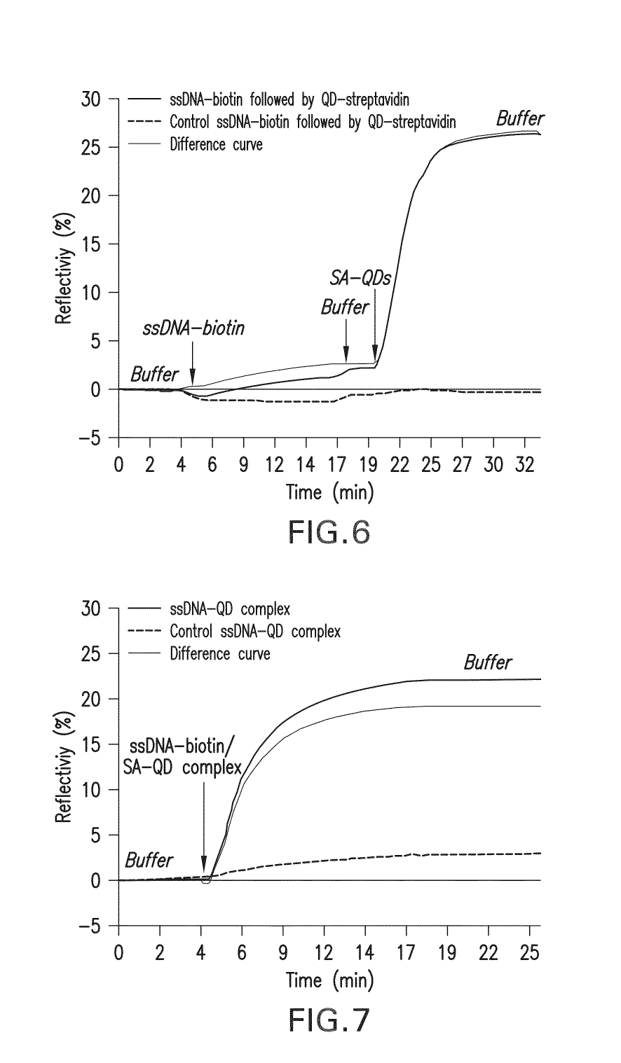

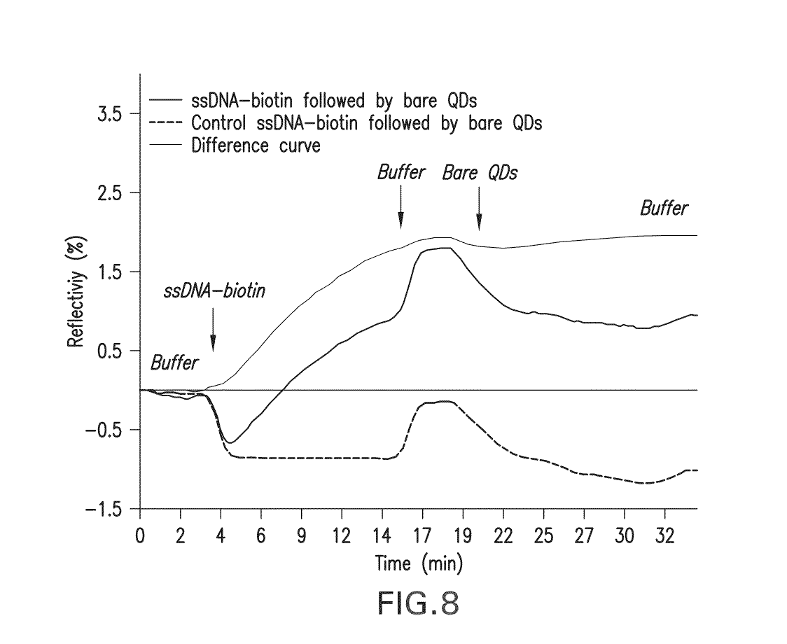

FIG. 6 shows a plot representation of the SPRi kinetic signal after the addition of SA-QDs to 50-biotin-tagged double-stranded DNA (dsDNA) target (solid) and nontargeted dsDNA (dashed), and the corresponding difference curve (dotted). FIG. 7 shows a plot representation of the SPRi kinetic signal after the addition of ssDNA-QD complex to thiol-modified ssDNA target sequence (black, solid) and nontargeted sequence (red, dashed), and the corresponding difference curve (dotted), and FIG. 8 bare NIR QDs to 50-biotin-tagged dsDNA target (solid) nontargeted dsDNA (dashed), and the corresponding difference curve (dotted).

FIGS. 4 and 5 illustrate methods according to the present invention for the detection of DNA hybridization using surface-immobilized ssDNA target sequence as a means of capture. FIG. 4 hybridizes a 5′-biotin-tagged complementary ssDNA sequence to the surface-immobilized ssDNA target sequence, followed by signal amplification with 5 nM streptavidin-coated QDs with 800 nm emission peak (SA-QDs, sandwich detection assay). FIG. 5 shows the use of 5′-biotin-tagged ssDNA sequences pre-functionalized to SA-QDs (direct detection assay). FIGS. 6 and 7 show the SPRi kinetic curves of the 50 nM ssDNA target binding to the immobilized probes for each of the implemented protocols. In both cases, a dramatic amplification in the signal is observed compared to the direct detection of the ssDNA target only. Upon the addition of the functionalized NIR QDs, approximately 10-fold signal amplification is observed for the direct detection assay (FIG. 7), while the SPR signal is amplified ˜15 times for the sandwich detection assay (FIG. 6). It is worth noting that the higher sensitivity of the latter assay (FIG. 6) is due to the increased detection specificity as demonstrated by the control curves (target binding to non-complementary probes) and the corresponding difference curves. The small drop in the signal following the injection of the DNA target is due to different salt concentrations between the DNA stock solution and the hybridization buffer, resulting in the negative change in the reflectivity for control. Therefore, the hybridization signal is taken as the difference between the initial and final buffer injections for the calculations of signal enhancement. In parallel, the nonspecific absorption of bare NIR QDs to the hybridized target is not significant (FIG. 8) and the bulk refractive index of the QD suspension does not contribute to the SPR signal amplification.

In a recent report, Okamato and colleagues observed a significant enhancement in emission intensities and decay when CdSe QDs were in close contact with evaporated gold films arising from surface plasmon (SP) coupling.²⁹ Since QDs have physical dimensions smaller than the exciton Bohr radius, their surface roughness near the metal layer increase considerably the coupling of the incident light to the surface plasmons.³⁰ Taking into account these findings and in addition to the mass-loading effect which is attributed to the high molecular weights or large size (15 to 20 nm) of the combination of QDs and streptavidin, the amplification mechanism of the SPRi signal by the bound QDs in the system according to one embodiment of the present invention is hypothesized to be due in part to the resonant coupling of QDs' spontaneous emission onto the propagating SPs. To validate this hypothesis, the mechanism of the signal amplification was subsequently investigated by assessing the influence of the QD peak emission wavelength (525, 705, 780 and 800 nm) on the SPRi signal enhancement.

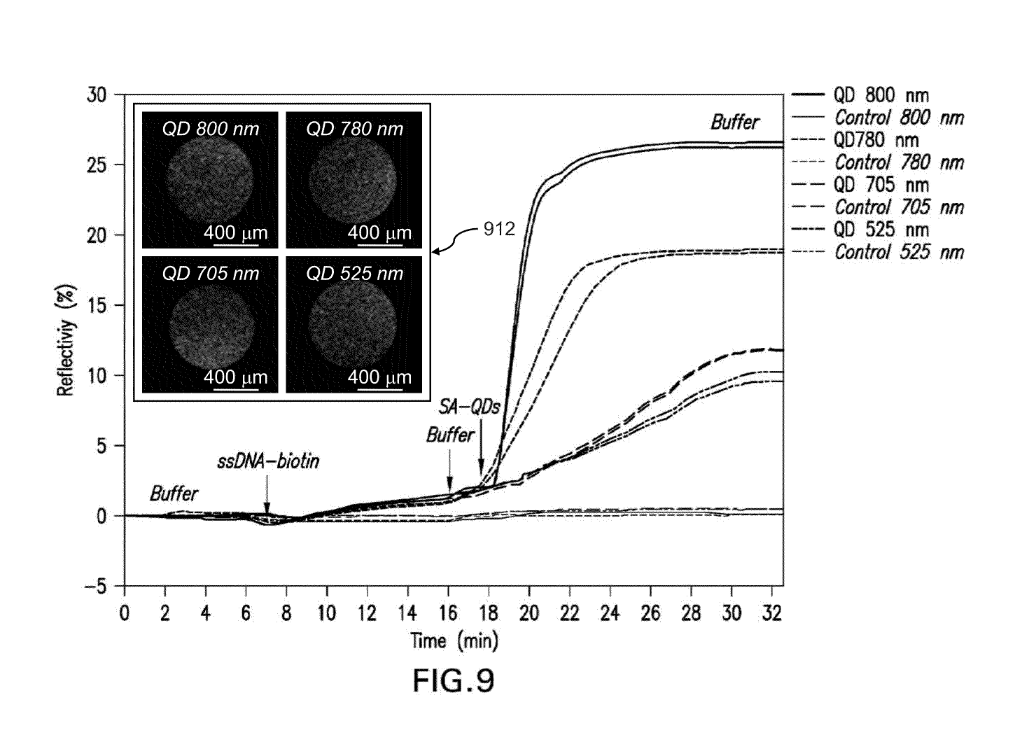

To confirm that the signal amplification is due in part to the enhanced SPs, QDs with emission peaks at 525, 705, 780 and 800 nm wavelengths were considered. From FIG. 9, it can be seen that the amplification is the most pronounced for the QD₈₀₀, whose emission peak matches the emission wavelength of the SPR source (the surface plasmon excitation wavelength). While the SPR signal largely depends on the surface functionalization of the QDs and their respective ability to bind to the biotinylated target, the comparison of the QD surface coverage via fluorescence imaging (inset 912 of FIG. 9) showed similar binding affinity for the different QDs used with uniform surface coverage. It is also noteworthy that the streptavidin-coated QDs emitting in the 525 to 800 nm range have estimated 15 to 20 nm diameters. Consequently, the SPR signal amplification is due in part to the mass-loading effect; however the significantly different signal enhancement between different QDs used herein cannot be attributed solely to the small difference in size (at most 5 nm between 800 nm QDs and 525 nm QDs). To further confirm this finding, a comparison between the 780 and 800 nm QDs was performed, due to the similarity in their size. By computing the signal enhancement as described above, the signal is amplified approximately 30% more for the 800 nm QDs. When the QD emission is set to the collective excitation band, it generates more of an effective and prevailing emission process from QDs to the free space via propagating SPs, thus surmounting the smaller scattering cross-section at the incident wavelength and generating a greater fluorescent enhancement.³¹ Since the QD₈₀₀ are tuned more towards the collective excitation band of the SPRi than the QD₇₈₀, a more efficient energy flow from QD₈₀₀ to the free space through SP scattering generates a greater change in reflectivity. Therefore, for all subsequent measurements we have employed QD₈₀₀ and sandwich assays for maximum SPRi signal enhancement.

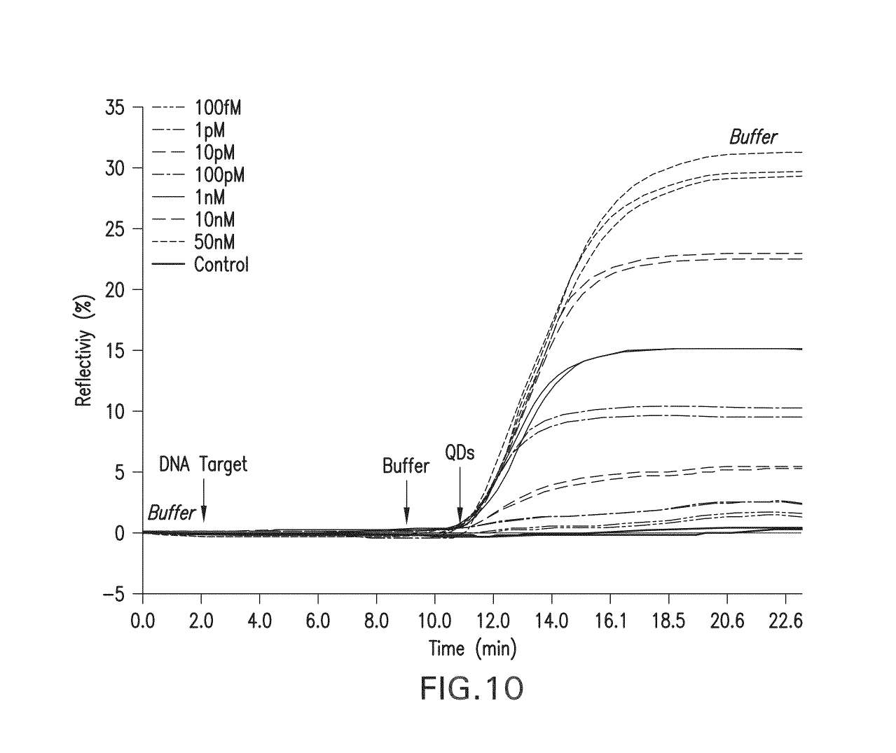

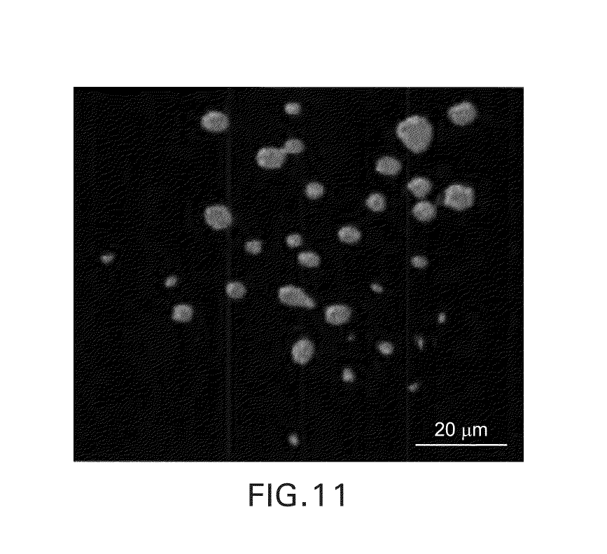

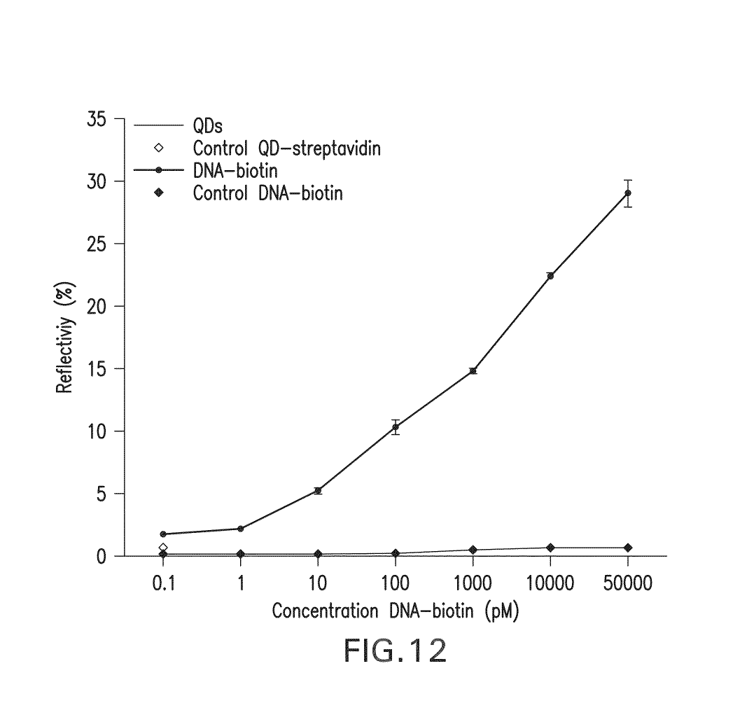

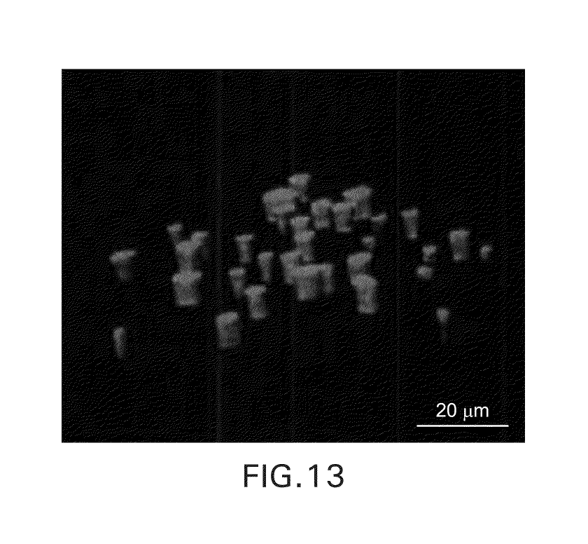

The detection of DNA hybridization using a sandwich assay was performed according to the protocol illustrated in FIG. 4. SPRi-binding kinetics for various concentrations of biotinylated DNA target sequence are shown in FIG. 10. FIG. 10 shows SPRi kinetic curves representing the detection of DNA hybridization using a sandwich assay for various ssDNA target concentrations. FIG. 11 shows concentration gradient curves representing the detection of DNA hybridization using a sandwich assay for various ssDNA target concentrations. FIG. 12 is a three-dimensional fluorescence image of the top view of an SPRi biochip after introduction of SA-QDs. FIG. 13 is a side view of the SPRi biochip of FIG. 12.

The target was injected into the flow cell for 8 minutes, followed by a buffer wash and a 10-minute incubation with 5 nM SA-QDs. The corresponding concentration gradient is plotted in FIG. 11 where each point represents the average value of the reflectivity difference between initial and final buffer injections calculated from three SPRi kinetic curves for each concentration. For comparison, the concentration gradient curve is also plotted for DNA hybridization assay without the amplification step. From FIG. 11, the LOD without the amplification step lies between 1 and 10 nM ssDNA complementary sequence concentration. Conversely, using the QD-amplification strategy, an improvement of more than six orders of magnitude in LOD and 25-fold signal enhancement (at the minimum detectable target concentration of 1 nM using the direct SPRi detection without amplification step) is obtained. The minimum detectable DNA concentration corresponded to 100 fM after an 8-minute hybridization reaction time. This LOD can be further reduced to 1 fM level for longer hybridization time (>4 hours). This system according to one embodiment of the present invention provides signal amplification with fM LOD as opposed to mM reported previously by Sendroiu et al.²⁰ In addition, QDs provide the means without any additional surface treatment to combine SPRi technology with fluorescence imaging. 3D images of the DNA sandwich assay biochip surface were reconstructed from z-sectional images and were loaded into the Imaris 6.3.1 imaging software program. FIGS. 12 and 13 show that the QDs are being localized on the surface of the biochip and the SPRi signal enhancement is due to their binding onto the surface.

### Example 2

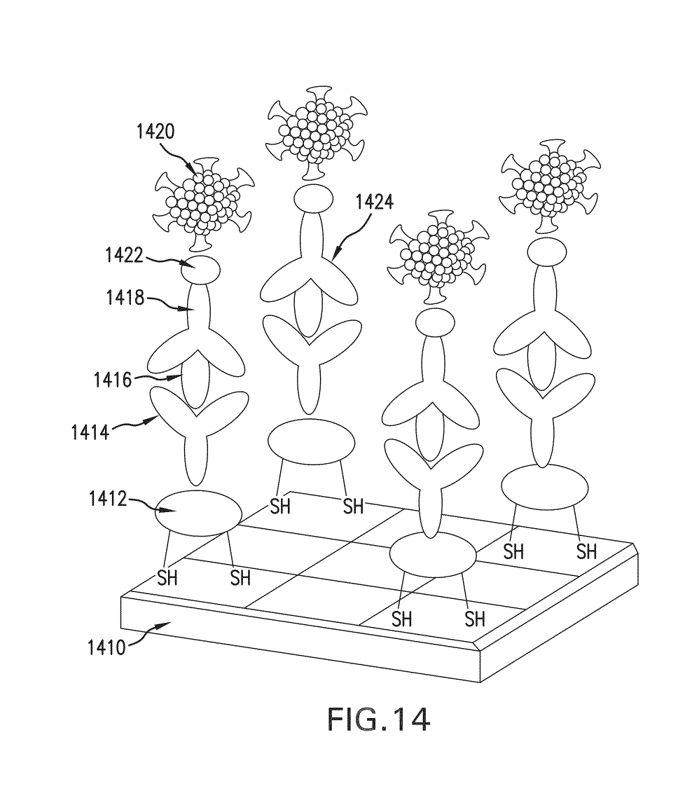

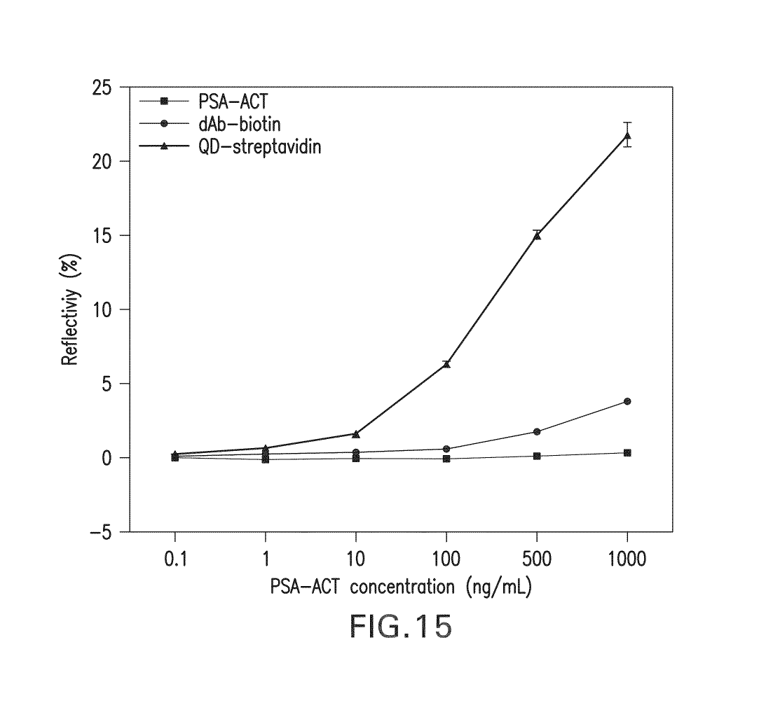

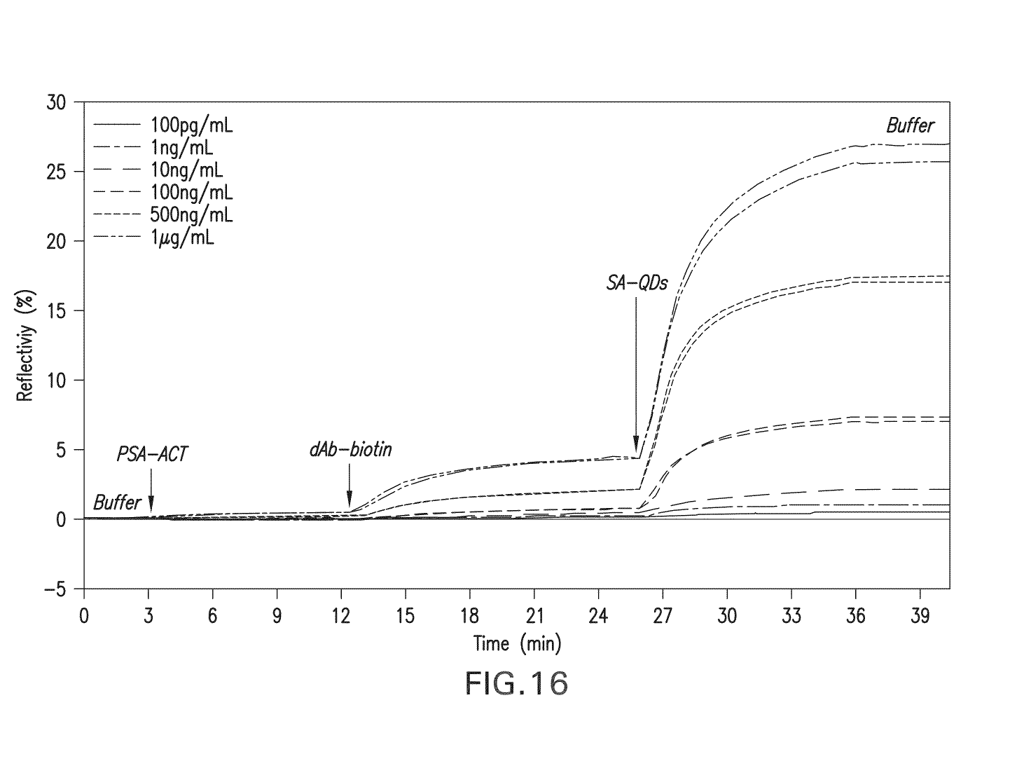

The versatility of the QD-enhanced SPRi detection is demonstrated by performing a SPRi immunoassay that targets a cancer biomarker prostate-specific-antigen (PSA bound to Alpha-1 Antichymotrypsin, PSA-ACT complex) as depicted in FIG. 14. FIG. 14 shows the functionalization of a gold chip 1410 functionalization with calixcrown ProLinker B 1412 followed by capture antibody PSA-ACT complex 1414, PSA-ACT antigen 1416, biotinylated detection antibody PSA-ACT complex 1418, and streptavidin-coated QDs 1420. Biotinylated detection antibody PSA-ACT complex 1418 includes biotin 1422 and detection antibody PSA-ACT complex 1424. FIG. 15 shows SPRi kinetic curves for various concentrations of PSA-ACT antigen and FIG. 16 shows the corresponding concentration gradient curves.

The sandwich assay is performed by the direct binding of the PSA-ACT antigen on Calixcrown-cAb functionalized gold surface for 10 minutes, followed by a first signal amplification with 20 μg/mL secondary biotinylated detection antibody (dAb-biotin) for 10 minutes, a second amplification using a 10-minute incubation with 5 nM SA-QDs and buffer wash steps in between each injection. The SPRi kinetic curves for various concentrations of PSA-ACT antigen are shown in FIG. 15, and the corresponding concentration gradient curves are plotted in FIG. 16. Each point of the curve represents the average of three measurements and is calculated by taking the SPRi signal difference between initial and final buffer injections. For comparison, the concentration gradient curve is also plotted for antibody-antigen binding without the amplification step and with a dAb-biotin amplification step. From FIG. 15 in the case of 1 μg/mL of antigen concentration, which corresponds to LOD for direct SPRi detection, the SPRi signal is amplified 50 times using a 10-minute QD incubation compared to the direct detection. The LOD for a direct binding assay lies between 0.5 and 1 μg/mL, while the LOD is reduced to 1 ng/mL for the dAb-biotin amplification step; the detection limit was further reduced to 100 pg/mL after QD amplification.

It this example, QD amplification appears to be less efficient in lowering the detection limit of the antigen detection in comparison to the DNA hybridization detection. This may be due to the differences in the binding efficiency for these two systems, as well as the differences in the size of surface-bound DNA and antigen/antibody complex, resulting in different vertical separation between QDs and the gold substrate which further affects the resonant coupling efficiency.

### Example 3

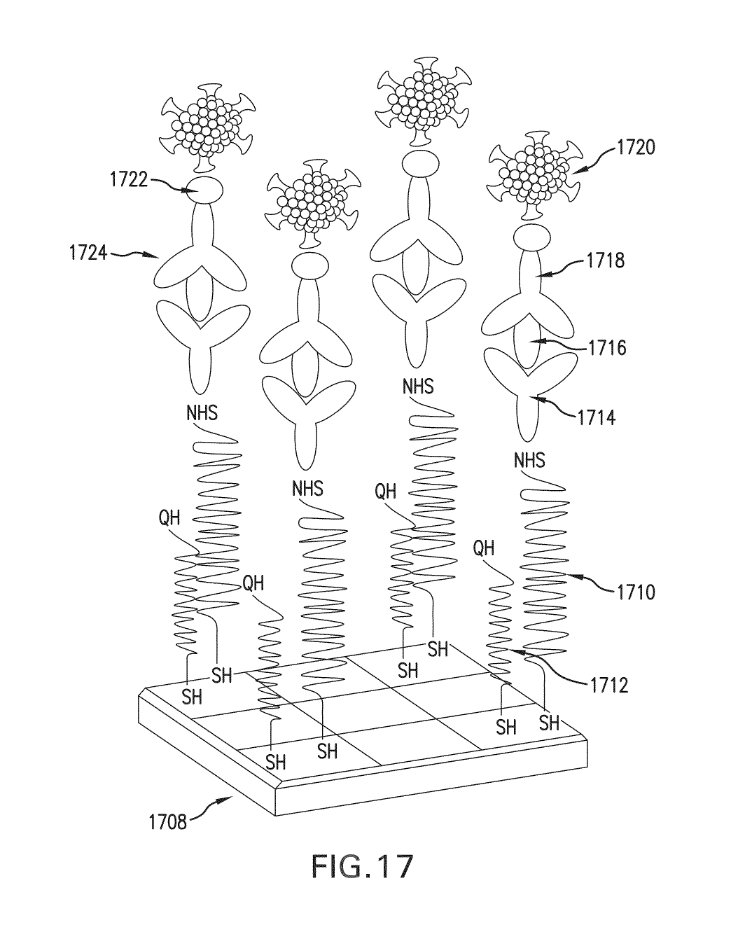

QD-enhanced SPRi detection of PSA at a clinically relevant concentration (2.5 ng/mL) in spiked serum (1:9 in HBS buffer) was performed to demonstrate the significance of the developed SPRi amplification strategy in potential diagnostic applications. Herein, the biosensor interface was functionalized with a mixture of thiolated PEG-COOH and PEG-OH (FIG. 17) due to the difficulty in reaching stable signals with the calixcrown surface chemistry at lower concentration levels (<10 ng/ml). FIG. 17 is a schematic representation of the functionalization of a gold chip 1708 with PEG-COOH 1710 and PEG-OH 1712 followed by the addition of capture antibody PSA-ACT complex 1714, PSA-ACT antigen 1716, biotinylated detection antibody PSA-ACT complex 1718, and streptavidin-coated QDs 1720. Biotinylated detection antibody PSA-ACT complex 1718 includes biotin 1722 and detection antibody PSA-ACT complex 1724.

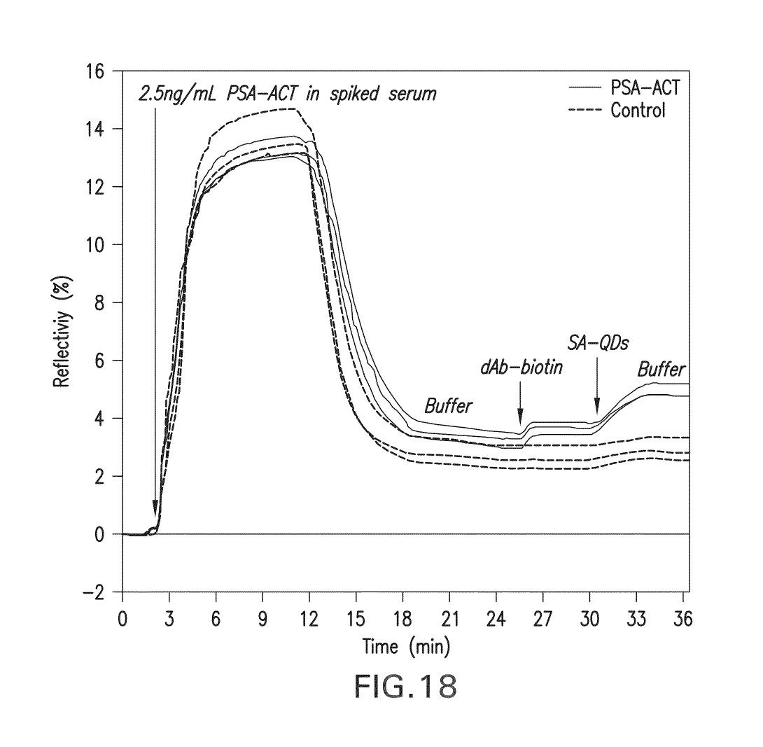

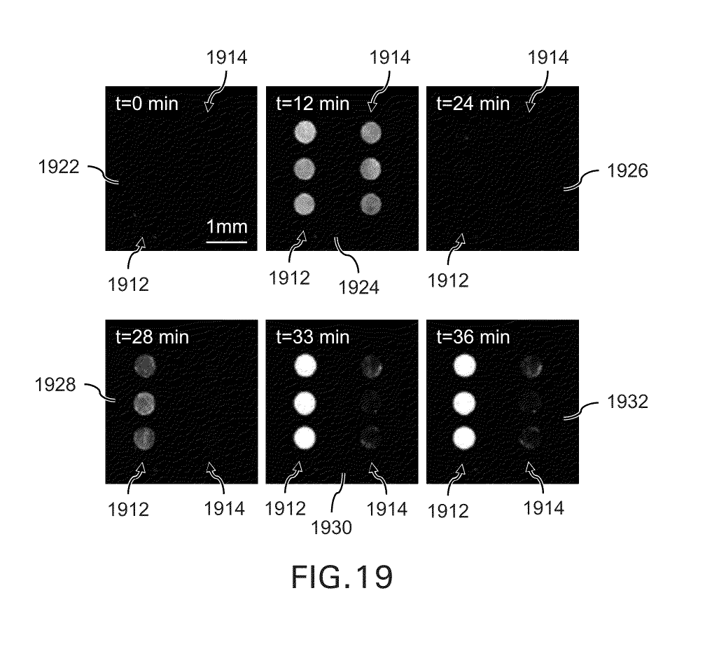

FIG. 18 is plot of SPRi kinetic curves for detection of PSA-ACT complex in spiked serum. FIG. 19 shows difference images corresponding to the curves of FIG. 18 showing time-lapsed binding kinetics for initial buffer injection, PSA-ACT complex in spiked serum injection, buffer wash, dAb-biotin injection, SA-QD injection, and final buffer wash on three spots of anti-PSA (left, indicated by arrow 1912) and anti-IgG (indicated by arrow 1914) at times 0 minutes (image 1922), 12 minutes (image 1924), 24 minutes (image 1926), 28 minutes (image 1928), 33 minutes (image 1930) and 36 minutes (image 1932). PSA-ACT complex in serum (2.5 ng/mL) was injected into the flow cell and allowed to bind to the thiolated PEG-COOH-cAb functionalized gold surface, followed by a 5-minute amplification step using 20 μg/mL dAb-biotin and 5 nM SA-QDs injections. From FIGS. 18 and 19, it can be seen that the direct antigen detection is incapable of distinguishing between the PSA antigen binding and control (anti-IgG), while the sandwich assay allows specific detection of the PSA-ACT complex at the concentration of interest. Additionally, the percent change in reflectivity after the addition of the SA-QDs was three times higher using the PEG-COOH/PEG-OH surface coating as opposed to the calixcrown surface coating (FIGS. 15 and 16) demonstrating that the SPRi signal amplification is also surface chemistry-dependent.

### Example 4

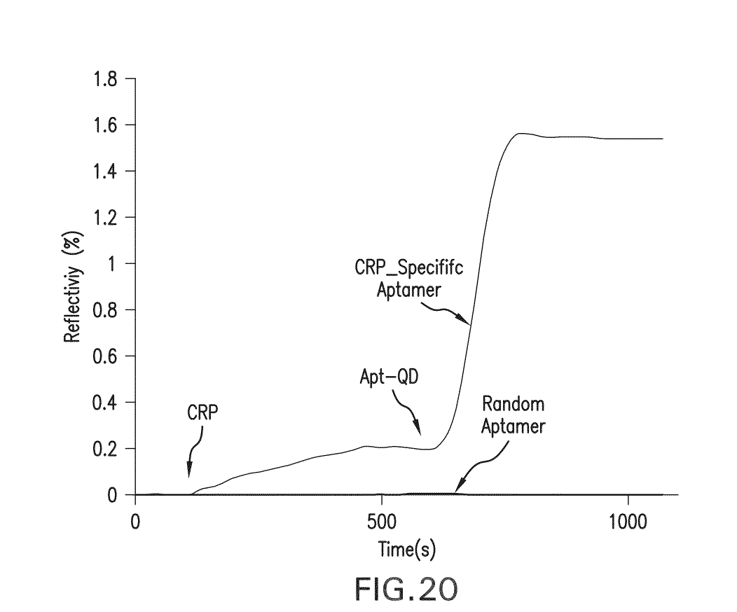

FIG. 20 is plot of SPRi kinetic curves for detection of C-reactive protein (CRP) in 10 mM Tris buffer containing 50 mM NaCl and 2 mM CaCl₂. In FIG. 20, one curve shows the reflectivity over time for a CRP-specific aptamer, while the other curve shows the reflectivity over time for a random (control) aptamer. 500 ng/ml of CRP spiked serum is added at time 107 seconds, as indicated by the arrow labeled “CRP.” At time 587 seconds, aptamer coated quantum dot complexes (Apt-QD) including a CRP-specific aptamer are added as indicated by the arrow labeled “Apt-QD.” As can be seen by the curve for the CRP-specific aptamer, the addition of Apt-QD results in a seven-fold SPRi signal enhancement, there was no enhancement observed for the random aptamer. The CRP-specific aptamer used in this example was 5′-/SThioMC6-D/GGC AGG AAG ACA AAC ACG ATG GGG GGGTAT GAT TTG ATGTGGTTG TTG CAT GAT CGT GGT CTGTGGTGC TGT-3′ (SEQ ID NO: 1). The random aptamer used in this example was 5-/SThioMC6-D/AGA AAA AAA AAC GCA AAA AAA AAA A -3 (SEQ ID NO: 2).

### Example 5

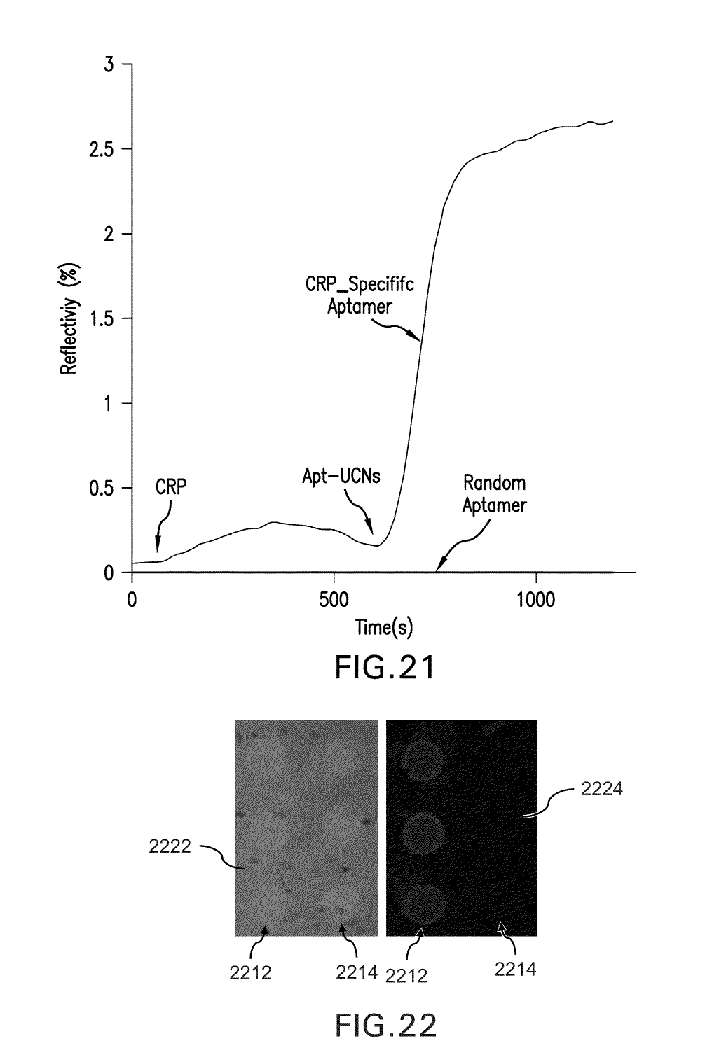

FIG. 21 is plot of SPRi kinetic curves for detection of C-reactive protein (CRP) in spiked serum. In FIG. 21, one curve shows the reflectivity over time for a CRP-specific aptamer, while the other curve shows the reflectivity over time for a random (control) aptamer. 500 ng/ml of CRP in 10 mM Tris buffer containing 50 mM NaCl and 2 mM CaCl₂ is added at time 47 seconds, as indicated by the arrow labeled “CRP.” At time 587 seconds, aptamer-coated Upconverting Nanoparticles complexes (Apt-UCNs) including a CRP-specific aptamer are added as indicated by the arrow labeled “Apt-UCNs.” As can be seen by the curve for the CRP-specific aptamer, the addition of Apt-UCNs results in a sixteen-fold SPRi signal enhancement. FIG. 22 shows difference images corresponding to the curves of FIG. 21 showing time-lapsed binding kinetics before injection of CRP and after injection of Apt-UCNs. Thiolated specific-CRP aptamers (left, indicated by arrow 2112) and thiolated random aptamers (indicated by arrow 2214) at times 0 seconds (image 2222). CRP complex in 10 mM Tris buffer containing 50 mM NaCl and 2 mM CaCl₂ (500 ng/mL) was injected into the flow cell and allowed to bind to the aptamer pre-functionalized gold surface resulting in a 17% change in reflectivity and then followed by the injection of Apt-UCNs (5 nM) at 547 seconds (left, indicated by arrow 2112 in image 2224) resulting in a 2.7% change in reflectivity (denoted by the change in contrast) and no binding was observed for the thiolated random aptamers (right, indicated by arrow 2114 in image 2224). The CRP-specific aptamer used in this example was 5′-/Biotin/GGC AGG AAG ACA AAC ACG ATG GGG GGGTAT GAT TTG ATGTGGTTG TTG CAT GAT CGT GGT CTGTGGTGC TGT-3′ (SEQ ID NO: 3). The random aptamer used in this example was 5-/SThioMC6-D/AGA AAA AAA AAC GCA AAA AAA AAA A-3 (SEQ ID NO: 4).

### REFERENCES

The following references are referred to above and are incorporated herein by reference:
1. K. T. Shimizu, W. K. Woo, B. R. Fisher, H. J. Eisler, M. G. Bawendi. Surface-Enhanced Emission from Single Semiconductor Nanocrystals. Phys. Rev. Lett., 89, 117401-04 (2002).
2. A. Imamoglu, D. D. Awschalom, G. Burkard, D. P. DiVincenzo, D. Loss, M. Sherwin, A. Small. Quantum information processing using quantum dot spins and cavity QED. Phys. Rev. Lett., 83, 4204-07 (1999).
3. S. Ulrich, S. Strauf, P. Michler. Triggered polarization-correlated photon pairs from a single CdSe quantum dot. Appl. Phys. Lett., 83, 1848-50 (2003).
4. R. Hanson, L. P. Kouwenhoven, J. R. Petta, S. Tarucha, L. M. K. Vandersypen. Spins in few-electron quantum dots. Rev. Mod. Phys., 79, 1217-65 (2007).
5. M. Schlamp, X. Peng, A. Alivisatos. Improved efficiencies in light emitting diodes made with CdSe(CdS) core/shell type nanocrystals and a semiconducting polymer. J. Appl. Phys., 82, 5837-42 (1997).
6. T. Pons, I. L. Medintz, K. E. Sapsford, S. Higashiya, A. F. Grimes, D. S. English, H. Mattoussi. On the Quenching of Semiconductor Quantum Dot Photoluminescence by Proximal Gold Nanoparticles. Nano Lett., 7, 3157-64 (2007).
7. M. G. Sandros, D. Gao, D. E. Benson. A Modular Nanoparticle-Based System for Reagentless Small Molecule Biosensing. J. Am. Chem. Soc., 127, 12198-99 (2005).
8. H. Wei, D. Ratchford, X. Li, H. Xu, C. Shih. Propagating Surface Plasmon Induced Photon Emission from Quantum Dots. Nano Lett., 9, 4168-71 (2009).
9. J. Homola. Surface Plasmon resonance sensors for detection of chemical and biological species. Chem. Rev., 108, 462-93 (2008).
10. E. J. Jeong, Y. S. Jeong, K. Park, S. Y. Yi, J. Ahn, S. J. Chung, M. Kim, B. H. Chung. Directed immobilization of DNA-binding proteins on a cognate DNA-modified chip surface. J. Biotechnol., 135, 16-21 (2008).
11. G. Hayashi, M. Hagihara, K. J. Nakatani. Genotyping by allele-specific 1-DNA-tagged PCR. J. Biotechnol., 135, 157-60 (2008).
12. B. H. Garcia II, R. M. Goodman. Use of surface plasmon resonance imaging to study viral RNA: protein interactions. J. Virol. Methods, 147, 18-25 (2008).
13. E. Mercey, R. Sadir, E. Maillart, A. Roget, F. Baleux, H. Lortat-Jacob, T. Livache. Polypyrrole oligosaccharide array and surface plasmon resonance imaging for the measurement of glycosaminoglycan binding interactions. Anal. Chem., 80, 3476-82 (2008).
14. M. Wakao, A. Saito, K. Ohishi, Y. Kishimoto, T. Nishimura, M. Sobel, Y. Suda. Sugar Chips immobilized with synthetic sulfated disaccharides of heparin/heparan sulfate partial structure. Bioorg. Med. Chem. Lett., 18, 2499-504 (2008).
15. E. Kretschmann, H. Raether. Radiative decay of non-radiative surface plasmons excited by light. Z. Naturforsch., 23, 2135-36 (1968).
16. C. E. Jordan, A. G. Frutos, A. J. Thiel, R. M. Corn. Surface Plasmon Resonance Imaging Measurements of DNA Hybridization Adsorption and Streptavidin/DNA Multilayer Formation at Chemically Modified Gold Surfaces. Anal. Chem., 69, 4939-47 (1997).
17. G. Q. Hu, Y. L. Gao, D. Q. Li. Modeling micropatterned antigen-antibody binding kinetics in a microfluidic chip. Biosens. Bioelectron., 22, 1403 (2007).
18. R. D'Agata, R. Corradini, G. Grasso, R. Marchelli, G. Spoto. Ultrasensitive Detection of DNA by PNA and Nanoparticle-Enhanced Surface Plasmon Resonance Imaging. ChemBioChem, 9, 2067-70 (2008).
19. S. Fang, H. J. Lee, A. W. Wark, R. M. Corn. Attomole microarray detection of microRNAs by nanoparticle-amplified SPR imaging measurements of surface polyadenylation reactions. J. Am. Chem. Soc., 128, 14044-46 (2006).
20. I. E. Sendroiu, M. E. Warner, R. M. Corn. Fabrication of silica-coated gold nanorods functionalized with DNA for enhanced surface plasmon resonance imaging biosensing applications. Langmuir, 25, 11282-84 (2009).
21. L. Malic, B. Cui, M. Tabrizian, T. Veres. Nanoimprinted plastic substrates for enhanced surface plasmon resonance imaging detection. Opt. Express, 17, 20386-92 (2009).
22. L. Malic, B. Cui, T. Veres, M. Tabrizian. Enhanced surface plasmon resonance imaging detection of DNA hybridization on periodic gold nanoposts. Opt. Lett., 32, 3092-94 (2007).
23. R. Robelek, L. Niu, E. L. Schmid, W. Knoll. Multiplexed hybridization detection of quantum dot-conjugated DNA sequences using surface plasmon-enhanced fluorescence microscopy and spectrometry. Anal. Chem., 76, 6160-65 (2004).
24. Q. Xiao, B. Zhou, S. Huang, F. Tian, H. Guan, Y. Ge, X. Liu, Z. He, Y. Liu. Direct observation of the binding process between protein and quantum dots by in situ surface plasmon resonance measurements. Nanotechnology, 20, 325101 (2009).
25. E. Golub, G. Pelossof, R. Freeman, H. Zhang, I. Willner. Electrochemical, photoelectrochemical, and surface plasmon resonance detection of cocaine using supramolecular aptamer complexes and metallic or semiconductor nanoparticles. Anal. Chem., 81, 9291 (2009).
26. L. Malic, T. Veres, M. Tabrizian. Nanostructured digital microfluidics for enhanced surface plasmon resonance imaging. Biosens. Bioelectron., 26, 2053-59 (2011).
27. Y. Lee, E. K. Lee, Y. W. Cho, T. Matsui, I. C. Kang, T. S. Kim, M. H. Han. ProteoChip: a highly sensitive protein microarray prepared by a novel method of protein immobilization for application of protein-protein interaction studies. Proteomics, 3, 2289-304 (2003).
28. C. Cao, J. P. Kim, B. W. Kim, H. Chae, H. C. Yoon, S. S. Yang, S. J. Sim. A strategy for sensitivity and specificity enhancements in prostate specific antigen-α1-antichymotrypsin detection based on surface plasmon resonance. Biosens. Bioelectron., 21, 2106-13 (2006).
29. K. Okamoto, S. Vyawahare, A. Scherer. Surface-plasmon enhanced bright emission from CdSe quantum-dot nanocrystals. J. Opt. Soc. Am. B, 23, 1674-78 (2006).
30. I. Gryczynski, J. Malicka, W. Jiang, H. Fischer, W. C. W. Chan, Z. Gryczynski, W. Grudzinski, J. R. Lakowicz. Surface-plasmon-coupled emission of quantum dots. J. Phys. Chem. B, 109, 1088-93 (2004).
31. J. H. Song, T. Atay, S. Shi, H. Urabe A. V. Nurmikko. Large enhancement of fluorescence efficiency from CdSe/ZnS quantum dots induced by resonant coupling to spatially controlled surface plasmons. Nano Lett., 5, 1557-61 (2005).

While the present invention has been disclosed with references to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method comprising the following steps:
(a) detecting binding of a streptavidin-coated quantum dot to an array of surface biomolecules to thereby produce detected binding results, and
(b) displaying the detected binding results to a user and/or saving the detected binding results to a storage medium,
wherein each surface biomolecule comprises an ssDNA hybrid that is bound to a gold substrate and a biotin-tagged ssDNA complementary sequence hybridized to a thiol-modified ssDNA probe sequence,
wherein the streptavidin-coated quantum dot binds to the biotin-tagged ssDNA complementary sequence, and
wherein step (a) comprises using surface plasmon resonance imaging on the gold substrate to detect the binding of the streptavidin-coated quantum dot to the array of surface biomolecules.

2. The method of claim 1, wherein step (b) comprises displaying the detected binding results to a user.

3. The method of claim 1, wherein step (b) comprises saving the detected binding results to a storage medium.

4. A method comprising the following steps:
(a) detecting binding of a plurality of ssDNA-protein-quantum dot complexes to an array of thiol-modified ssDNA probe sequences to thereby produce detected binding results, and
(b) displaying the detected binding results to a user and/or saving the detected binding results to a storage medium,
wherein the thiol-modified ssDNA probe sequences are bound to a gold substrate,
wherein each ssDNA-protein-quantum dot complex comprises:
a streptavidin-coated quantum dot,
one or more biotin-tagged ssDNA complementary sequences bound to respective streptavidin-coated quantum dots of the one or more streptavidin-coated quantum dots,
wherein each of the ssDNA-protein-quantum dot complexes binds to a respective thiol-modified ssDNA probe sequence of the array of thiol-modified ssDNA probe sequences, and
wherein step (a) comprises using surface plasmon resonance imaging on the gold substrate to detect the binding of the plurality of ssDNA-protein-quantum dot complexes to the array of thiol-modified ssDNA probe sequences.

5. The method of claim 4, wherein step (b) comprises displaying the detected binding results to a user.

6. The method of claim 4, wherein step (b) comprises saving the detected binding results to a storage medium.

7. A composition comprising:
a capture antibody complex,
an antigen bound to the capture antibody complex, and
a biotinylated-detection antibody complex bound to the antigen.

8. The composition of claim 7, wherein the composition comprises a streptavidin-coated quantum dot bound to the detection antibody complex.

9. The composition of claim 7, wherein the composition comprises an avidin-coated quantum dot bound to the detection antibody complex.

10. The composition of claim 7, wherein the capture antibody complex comprises a prostate-specific antigen antichymotrypsin (PSA-ACT) complex, the antibody comprises PSA-ACT antigen and the detection antibody complex comprises a biotinylated detection antibody PSA-ACT complex.

11. The composition of claim 7, wherein the capture complex comprises of thiolated specific-CRP aptamer, a CRP protein and a CRP-specific aptamer coated on a nanoparticle.

12. A method comprising the following steps:
(a) detecting binding of a plurality of biomarkers to an array of surface biomolecules to thereby produce detected binding results, and
(b) displaying the detected binding results to a user and/or saving the detected binding results to a storage medium,
wherein each surface biomolecule comprises a calixcrown ProLinker B bound to a gold substrate,
wherein each biomarker comprises:
a capture antibody complex, and
an antigen bound to the capture antibody complex,
wherein a biotinylated-detection antibody complex is bound to the antigen,
wherein a streptavidin-coated quantum dot is bound to the biotinylated-detection antibody complex, and
wherein step (a) comprises using surface plasmon resonance imaging on the gold substrate to detect the binding of the plurality of biomarkers to the array of surface biomolecules.

13. The method of claim 12, wherein step (b) comprises displaying the detected binding results to a user.

14. The method of claim 12, wherein step (b) comprises saving the detected binding results to a storage medium.

15. The method of claim 12, wherein the method comprises the following steps prior to step (a):
(c) exposing the plurality of biomarkers bound to the array of surface biomolecules to the biotinylated-detection antibody complex to bind the biotinylated-detection antibody complex to the biomarkers to thereby form a plurality of biotinylated biomarkers, and
(d) exposing the plurality of biotinylated biomarkers to streptavidin-coated quantum dots to bind the streptavidin-coated quantum dots to the biotinylated biomarkers.

16. The method of claim 15, wherein prior to step (c) the method comprises the following step:
(e) binding the plurality of biomarkers to an array of surface biomolecules.

17. The method of claim 12, wherein the capture antibody complex comprises a prostate-specific antigen antichymotrypsin (PSA-ACT) complex, the antibody comprises PSA-ACT antigen and the detection antibody complex comprises a biotinylated detection antibody PSA-ACT complex.

18. The method of claim 12, wherein the capture antibody complex comprises a thiolated specific-CRP aptamer, a CRP protein and a CRP-specific aptamer coated on a nanoparticle.

19. A method comprising the following steps:
(a) detecting binding of a plurality of biomarkers to an array of surface biomolecules to thereby produce detected binding results, and
(b) displaying the detected binding results to a user and/or saving the detected binding results to a storage medium,
wherein each surface biomolecule comprises a thiolated polyethylene glycol bound to a gold substrate,
wherein each biomarker comprises:
a capture antibody complex, and
an antigen bound to the capture antibody complex,
wherein a biotinylated-detection antibody complex is bound to the antigen,
wherein a streptavidin-coated quantum dot is bound to the biotinylated-detection antibody complex, and
wherein step (a) comprises using surface plasmon resonance imaging on the gold substrate to detect the binding of the plurality of biomarkers to the array of surface biomolecules.

20. The method of claim 19, wherein step (b) comprises displaying the detected binding results to a user.

21. The method of claim 19, wherein step (b) comprises saving the detected binding results to a storage medium.

22. The method of claim 19, wherein the method comprises the following steps prior to step (a):
(c) exposing the plurality of biomarkers bound to the array of surface biomolecules to the biotinylated-detection antibody complex to bind the biotinylated-detection antibody complex to the biomarkers to thereby form a plurality of biotinylated biomarkers, and
(d) exposing the plurality of biotinylated biomarkers to streptavidin-coated quantum dots to bind the streptavidin-coated quantum dots to the biotinylated biomarkers.

23. The method of claim 22, wherein prior to step (c) the method comprises the following step:
(e) binding the plurality of biomarkers to an array of surface biomolecules.

24. The method of claim 19, wherein capture antibody complex comprises a prostate-specific antigen antichymotrypsin (PSA-ACT) complex, the antibody comprises PSA-ACT antigen and the detection antibody complex comprises a biotinylated detection antibody PSA-ACT complex.

25. The method of claim 19, wherein the capture antibody complex comprises a thiolated specific-CRP aptamer, a CRP protein and a CRP-specific aptamer coated on a nanoparticle.

26. A method comprising the following steps:
(a) detecting binding of an avidin-coated quantum dot to an array of surface biomolecules to thereby produce detected binding results, and
(b) displaying the detected binding results to a user and/or saving the detected binding results to a storage medium,
wherein each surface biomolecule comprises an ssDNA hybrid that is bound to a gold substrate and a biotin-tagged ssDNA complementary sequence hybridized to a thiol-modified ssDNA probe sequence,
wherein the avidin-coated quantum dot binds to the biotin-tagged ssDNA complementary sequence, and
wherein step (a) comprises using surface plasmon resonance imaging on the gold substrate to detect the binding of the avidin-coated quantum dot to the array of surface biomolecules.

27. The method of claim 26, wherein step (b) comprises displaying the detected binding results to a user.

28. The method of claim 26, wherein step (b) comprises saving the detected binding results to a storage medium.

29. A method comprising the following steps:
(a) detecting binding of a plurality of ssDNA-protein-quantum dot complexes to an array of thiol-modified ssDNA probe sequences to thereby produce detected binding results, and
(b) displaying the detected binding results to a user and/or saving the detected binding results to a storage medium,
wherein the thiol-modified ssDNA probe sequences are bound to a gold substrate,
wherein each ssDNA-protein-quantum dot complex comprises:
an avidin-coated quantum dot,
one or more biotin-tagged ssDNA complementary sequences bound to respective avidin-coated quantum dots of the one or more avidin-coated quantum dots,
wherein each of the ssDNA-protein-quantum dot complexes binds to a respective thiol-modified ssDNA probe sequence of the array of thiol-modified ssDNA probe sequences, and
wherein step (a) comprises using surface plasmon resonance imaging on the gold substrate to detect the binding of the plurality of ssDNA-protein-quantum dot complexes to the array of thiol-modified ssDNA probe sequences.

30. The method of claim 29, wherein step (b) comprises displaying the detected binding results to a user.

31. The method of claim 29, wherein step (b) comprises saving the detected binding results to a storage medium.

32. A method comprising the following steps:
(a) detecting binding of a plurality of biomarkers to an array of surface biomolecules to thereby produce detected binding results, and
(b) displaying the detected binding results to a user and/or saving the detected binding results to a storage medium,
wherein each surface biomolecule comprises a calixcrown ProLinker B bound to a gold substrate,
wherein each biomarker comprises:
a capture antibody complex, and
an antigen bound to the capture antibody complex,
wherein a biotinylated-detection antibody complex is bound to the antigen,
wherein an avidin-coated quantum dot is bound to the biotinylated-detection antibody complex, and
wherein step (a) comprises using surface plasmon resonance imaging on the gold substrate to detect the binding of the plurality of biomarkers to the array of surface biomolecules.

33. The method of claim 32, wherein step (b) comprises displaying the detected binding results to a user.

34. The method of claim 32, wherein step (b) comprises saving the detected binding results to a storage medium.

35. The method of claim 32, wherein the method comprises the following steps prior to step (a):
(c) exposing the plurality of biomarkers bound to the array of surface biomolecules to the biotinylated-detection antibody complex to bind the biotinylated-detection antibody complex to the biomarkers to thereby form a plurality of biotinylated biomarkers, and
(d) exposing the plurality of biotinylated biomarkers to avidin-coated quantum dots to bind the avidin-coated quantum dots to the biotinylated biomarkers.

36. The method of claim 35, wherein prior to step (c) the method comprises the following step:
(e) binding the plurality of biomarkers to an array of surface biomolecules.

37. The method of claim 32, wherein capture antibody complex comprises a prostate-specific antigen antichymotrypsin (PSA-ACT) complex, the antibody comprises PSA-ACT antigen and the detection antibody complex comprises a biotinylated detection antibody PSA-ACT complex.

38. The method of claim 32, wherein the capture antibody complex comprises a thiolated specific-CRP aptamer, a CRP protein and a CRP-specific aptamer coated on a nanoparticle.

39. A method comprising the following steps:
(a) detecting binding of a plurality of biomarkers to an array of surface biomolecules to thereby produce detected binding results, and
(b) displaying the detected binding results to a user and/or saving the detected binding results to a storage medium,
wherein each surface biomolecule comprises a thiolated polyethylene glycol bound to
a gold substrate,
wherein each biomarker comprises:
a capture antibody complex, and
an antigen bound to the capture antibody complex,
wherein a biotinylated-detection antibody complex is bound to the antigen,
wherein an avidin-coated quantum dot is bound to the biotinylated-detection antibody complex, and
wherein step (a) comprises using surface plasmon resonance imaging on the gold substrate to detect the binding of the plurality of biomarkers to the array of surface biomolecules.

40. The method of claim 39, wherein step (b) comprises displaying the detected binding results to a user.

41. The method of claim 39, wherein step (b) comprises saving the detected binding results to a storage medium.

42. The method of claim 39, wherein the method comprises the following steps prior to step (a):
(c) exposing the plurality of biomarkers bound to the array of surface biomolecules to the biotinylated-detection antibody complex to bind the biotinylated-detection antibody complex to the biomarkers to thereby form a plurality of biotinylated biomarkers, and
(d) exposing the plurality of biotinylated biomarkers to avidin-coated quantum dots to bind the avidin-coated quantum dots to the biotinylated biomarkers.

43. The method of claim 42, wherein prior to step (c) the method comprises the following step:
(e) binding the plurality of biomarkers to an array of surface biomolecules.

44. The method of claim 39, wherein capture antibody complex comprises a prostate-specific antigen antichymotrypsin (PSA-ACT) complex, the antibody comprises PSA-ACT antigen and the detection antibody complex comprises a biotinylated detection antibody PSA-ACT complex.

45. The method of claim 39, wherein the capture antibody complex comprises a thiolated specific-CRP aptamer, a CRP protein and a CRP-specific aptamer coated on a nanoparticle.
